(19) United States
(12) Patent Application Publication
(10) Pub. No.: US 2022/0338786 A1
(43) Pub. Date: Oct. 27, 2022

(54) PERIOD-BASED ARTIFACT RECONSTRUCTION AND REMOVAL FOR DEEP BRAIN STIMULATION

(71) Applicant: BROWN UNIVERSITY, Providence, RI (US)

(72) Inventors: David A. Borton, Pawtucket, RI (US); Evan M. Dastin-van Rijn, Silver Spring, MD (US); Matthew T. Harrison, Providence, RI (US); Nicole R. Provenza, Providence, RI (US); Paula Chen, Storrs, CT (US); Taewoo Kim, Providence, RI (US); Jerome Darbon, Providence, RI (US)

(21) Appl. No.: 17/722,903

(22) Filed: Apr. 18, 2022

Related U.S. Application Data
(60) Provisional application No. 63/175,838, filed on Apr. 16, 2021.
(60) Provisional application No. 63/184,891, filed on May 6, 2021.

(51) Int. Cl. A61B 5/37; A61B 5/372; A61B 5/00

(52) U.S. Cl. CPC A61B 5/37; A61B 5/372; A61B 5/7203; A61B 5/725

(57) ABSTRACT

Methods and systems for improved removal of deep brain stimulation artifacts from electrical measurements of brain activity. In one embodiment, a method is provided that includes: receiving, by a computing device having a processor, waveform data caused by a deep brain stimulation of a patient-specific area of interest; determining, by the computing device, a stimulation period relative to a sampling rate; identifying, by the computing device and based on the waveform data and the stimulation period, a stimulation artifact; subtracting, by the computing device, the identified stimulation artifact from the received waveform data; and generating, in real-time, a filtered waveform data indicating an absence of the stimulation artifact.

### RELATED APPLICATIONS

The present application claims priority to and the benefit of U.S. Provisional Application 63/175,838, filed Apr. 16, 2021, and U.S. Provisional Application 63/184,891, filed May 6, 2021, the entireties of which are hereby incorporated herein by reference.

### STATEMENT OF GOVERNMENT RIGHTS

This invention was made with government support under Grant Number NS100549 awarded by the National Institutes of Health. The government has certain rights in the invention.

### BACKGROUND

Deep brain stimulation (DBS) has proven to be effective in treating several neurological disorders, including Parkinson's Disease, epilepsy, and Obsessive Compulsive Disorder. Advances in device development over the last decade have enabled concurrent stimulation and recording at adjacent locations in the brain. These chronic neural recordings present an opportunity for identification of neural signatures of behaviors relevant to disease states. However, DBS and other electrical stimulations creates high amplitude, high frequency stimulation artifacts that contaminate the electrical recordings of the brain. As a result, there is a need for effective periodic artifact removal systems and methods for recovering the underlying neural signal and thus, for understanding the biological mechanisms underlying the qualitative successes of DBS.

In DBS applications, the artifact removal problem is complicated by a variety of factors. For example, while the stimulation frequency can be set by the DBS device, the device setting provides an estimate of the stimulation frequency (and period) that is not accurate enough for successful artifact removal. Furthermore, the amplitude of DBS artifacts are often orders of magnitude larger than the underlying neural activity, and the sampling rate of the neural recordings is often low compared to the high frequency of therapeutic DBS. For example, power constraints of DBS devices often require low sampling rates (e.g., 200-250 Hz), which in turn alias the stimulation frequency near the frequencies of underlying signals of interest. In addition, DBS recordings are often broken into many time segments with unknown phases, e.g., due to modulation of device settings or missing data of unknown length, the latter of which is a common limitation of the wireless data transmission methods used by some DBS devices.

There is a desire and need for computationally efficient artifact removal systems and methods that are effective in removing periodic artifacts from DBS and other electrical stimulation recordings (e.g., sampled at less than twice the frequency of DBS). Moreover, as periodic artifacts in DBS recordings may often cause multiple unknown phase shifts, there is a desire and need for computationally efficient estimation of periodic artifacts in the presence of such phase shifts. Furthermore, there is a desire and need for systems and methods of recovering and/or assessing the scale of lost packets during transmission of waveform data produced by DBS and other electrical stimulation. This assessment of lost packets may facilitate data alignment in the artifact removal process and in the recovery of neural signals necessary for, e.g. control of therapy.

Various embodiments of the present disclosure describe systems and methods that address one or more of the above described shortcomings.

### SUMMARY

The present disclosure presents new and innovative systems and methods for identifying and removing DBS artifacts from electrical measurements of brain activity. The system may perform several steps or processes to identify and remove DBS artifacts, including (1) generating an initial guess for a period of artifacts created by a DBS therapy signal, (2) determining the true period for the artifacts created by the DBS therapy signal based on the initial guess, and removing artifacts based on the true period.

In at least one embodiment, a system for stimulation period-based artifact removal and reconstruction is disclosed. The system may include intracranial electroencephalography (iEEG) device; one or more processors; and memory. The memory may store instructions that, when executed by the one or more processors, cause the one or more processors to: receive, in real-time from the iEEG device, waveform data caused by a deep brain stimulation of a patient-specific area of interest; and determine a stimulation period relative to a sampling rate. The stimulation period may be determined using an iterative (e.g., stage-wise) process, and may utilize regulation of the waveform data. The instructions may further cause the one or more processor to identify, by applying Nadaraya-Watson kernel regression and a linear filter to the waveform data and the stimulation period, a stimulation artifact; subtract the identified stimulation artifact from the received waveform data; and generate, in real-time, a filtered waveform data indicating an absence of the stimulation artifact.

In some embodiments, the instructions, when executed, may further cause the one or more processors to determine the stimulation period by identifying, between two contiguous time segments of the waveform data, one or more phase shifts in the waveform data; estimating, based on the one or more phase shifts, a number of time points in one or more missing packets of unknown duration; and realigning, based on the number of time points in the one or more missing packets, the waveform data. The stimulation period may be based on the realigned waveform data.

In another embodiment, a method of period-based estimation of electrical stimulation artifacts in the presence of phase shifts is disclosed. The method may include: receiving, in real-time by a computing device having a processor, waveform data caused by a deep brain stimulation of a patient-specific area of interest. The waveform data may comprise: a plurality of runs, a plurality of phase shifts, and a periodic artifact. Each run may indicate a contiguous portion of the waveform data separated by a packet loss. The method may further include generating a model of the received waveform data based on: a neural signal of interest and a model of the periodic artifact. The model of the periodic artifact may be based on a stimulation period of the periodic artifact and a phase shift of the plurality of phase shifts. For example, a model of the periodic artifact may be defined as:

$${S}_{i} ( t ) = A ( t + \frac{{δ}_{i}^{*}}{{ξ}^{*}} ) + {B}_{i} ( t ) + {η}_{i} ( t ) , i = 0 , … , n ,$$

where δᵢ* comprises the phase shift, of the plurality of phase shifts, between the 0th and i-th run of the plurality of runs, A is the model of the periodic artifact based on a stimulation period

$$( \frac{1}{{ξ}^{*}} )$$

of the periodic artifact and the phase shift (δᵢ*) of the plurality of phase shifts, Bᵢ(t) is the neural signal of interest at the i-th run, and ηᵢ(t) is noise at the i-th run. The method may further include applying harmonic regression to optimize the model of the periodic artifact. In some embodiments, the model of the periodic artifact to which harmonic regression is applied may be characterized as a(t|ξ,δ,α₀,αₖ,βₖ,K)=α₀+Σₖ₌₁^{K}(αₖ cos(2πk(ξt+δ))+βₖ sin(2πk(ξt+δ))). wherein K is a finite number of harmonics, k. The method may further include determining simultaneously, using an optimization of a loss model, the plurality of phase shifts and the neural signal of interest, wherein the loss model is based on the model of the received waveform data and an optimized model of the periodic artifact. For example, the loss function model may be characterized as L(ξ,δᵢ,θ)=Σᵢ₌₀ⁿΣ_{tϵT}_{ᵢ}(Sᵢ(t)−α(t,ξ,δᵢ,θ))², where α(·|ξ,δᵢ,θ) is the optimized model of the periodic artifact, where the optimized model of the periodic artifact is based on: a stimulation frequency of the periodic artifact, the phase shift (Sᵢ of the plurality of phase shifts, and one or more parameters for the periodic artifact; and where Sᵢ(t) is the model of the received waveform data at time t, and sampled at discrete times, Tᵢ.

In another embodiment, the present disclosure presents new and innovative systems and methods for improved packet loss estimation. In one aspect a method is provided that includes dividing time series data into a plurality of runs. A period of stimulation may be estimated from data in the plurality of runs. A harmonic regression model may be fit to a longest run of the plurality of runs and packet loss may be estimated based on the harmonic regression mode.

In another aspect, a method for periodic estimation of lost packets included: receiving, in real-time by a computing device having a processor, waveform data caused by a deep brain stimulation of a patient-specific area of interest. The waveform data comprises a plurality of runs. Each run may indicate a contiguous portion of the waveform data separated by a packet loss. The method may further include: determining, by the computing device, a stimulation period relative to a sampling rate. Furthermore, the computing device may apply, based on the stimulation period, a harmonic regression model to a longest run of the plurality of runs; and determine, in real-time and based on the harmonic regression model, an optimal size of packet loss for the waveform data.

The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the disclosed subject matter.

### BRIEF DESCRIPTION OF THE FIGURES

FIG. 1 illustrates an example computer system according to a non-limiting embodiment of the present disclosure.

FIG. 2 is a schematic illustrating the detection of artifacts during deep brain stimulation, according to a non-limiting embodiment of the present disclosure.

FIG. 3 is a flow diagram showing an example method of deep brain stimulation artifact identification and removal, according to a non-limiting embodiment of the present disclosure.

FIG. 4 is a flow diagram showing an example method of determining a stimulation period relative to a sampling rate, according to a non-limiting embodiment of the present disclosure.

FIG. 5 is a schematic illustrating stimulation period determination, stimulation artifact reconstruction, and stimulation artifact removal, according to a non-limiting embodiment of the present disclosure.

FIG. 6 shows example spectrograms and charts illustrating the recovery of sinusoidal signals during deep brain stimulation artifact identification and removal, according to a non-limiting embodiment of the present disclosure.

FIG. 7 shows computer simulations comparing the deep brain stimulation artifact identification and removal to conventional filters, according to a non-limiting embodiment of the present disclosure.

FIG. 8 is a schematic illustrating packet loss in waveform data from deep brain stimulation, according to a non-limiting embodiment of the present disclosure.

FIG. 9 is a flow diagram showing an example method of a stimulation period based estimation of packet loss, according to a non-limiting embodiment of the present disclosure.

FIG. 10 is a flow diagram showing an example method of applying a harmonic regression model to estimate packet loss, according to a non-limiting embodiment of the present disclosure.

FIG. 11 is a schematic illustrating a stimulation period-based estimation of packet loss, according to a non-limiting embodiment of the present disclosure.

FIG. 12 shows sinograms illustrating the fitting of a stimulation model to waveform data, according to a non-limiting embodiment of the present disclosure.

FIG. 13 shows graphs illustrating results of a stimulation model, according to a non-limiting embodiment of the present disclosure.

FIG. 14 shows Monte Carlo simulated packet loss, according to a non-limiting embodiment of the present disclosure.

FIG. 15 shows graphs illustrating packet loss over a stimulation period, according to a non-limiting embodiment of the present disclosure.

FIG. 16 is a flow diagram showing an example method of period-based estimation of electrical stimulation artifacts in the presence of phase shifts, according to a non-limiting embodiment of the present disclosure.

FIG. 17 shows graphs illustrating the effectiveness of the method of period-based estimation of electrical stimulation artifacts in the presence of phase shifts, according to a non-limiting embodiment of the present disclosure.

### DETAILED DESCRIPTION OF EXAMPLE EMBODIMENTS

Today, state-of-the-art artifact removal methods from electrical stimulation therapies (e.g., deep brain stimulation (DBS) may often rely on template subtraction of the artifact at each electrical stimulation (e.g., DBS) pulse. However, existing methods for identifying individual pulses (e.g. thresholding) are not robust for low sampling rates or in the presence of other spurious high amplitude artifacts. Various embodiments of the present disclosure describe a novel and nonobvious systems and methods that identify each instance of the artifact by windowing the signal based on the exact period of stimulation. Finding the true period is not straightforward due to discrepancies between reported sampling rate and stimulation frequency of existing DBS and other electrical stimulation devices. There is a desire and need for systems and methods of finding the true period of stimulation. The presently disclosed systems and methods effectively and efficiently find the true period of any periodic signal, including that of DBS artifact. After the true period is found, a template of the artifact can be reconstructed and subtracted at each instance of the DBS artifact without contaminating the underlying signal of interest. The presently disclosed system and method is robust for non-periodic spurious high amplitude artifacts and for low sampling rates. The recovered underlying neural signal of interest is not contaminated even when the frequency content of the aliased artifact and underlying neural signal overlaps. Additionally, the presently disclosed systems and methods are computationally efficient and could be implemented onboard existing or future DBS devices to remove DBS artifacts in real time. The present disclosure also provides artifact removal methods that are effective in removing periodic artifacts from local field potential (LFP) and other electrical stimulation recordings sampled at less than twice the frequency of the electrical field stimulation (e.g., DBS).

The presently disclosed systems and methods can be used by DBS device companies and researchers to implement offline or onboard artifact subtraction to enable biomarker detection during concurrent sensing and DBS therapy. Additionally, the presently disclosed systems and methods could be used in other fields to recover any type of signal with a period artifact. These techniques are discussed in further detail below.

High amplitude and high frequency DBS artifacts complicate biomarker detection necessary for the development of adaptive DBS therapy. DBS artifacts can be difficult to remove, especially in low sampling rate recordings where components of the artifact are aliased. Existing methods rely on finding individual DBS pulses via thresholding which may be ineffective for the low sampling rates and long artifacts characteristic of electrical stimulation recordings (e.g., LFP)

Various embodiments of the present disclosure describe a novel and nonobvious method (referred to herein as period-based artifact reconstruction and removal method (PARRM)), and systems for implementing the same, which improve on existing methodologies by accurately finding each instance of the artifact. Experiments and results described herein demonstrate that PARRM is effective in removing high frequency DBS artifact from relatively low sample rate electrical stimulation recordings (e.g., DBS recordings, LFP recordings, etc.), without introducing contamination to the underlying neural signal.

Biomarker detection requires a hardware platform capable of concurrent sensing and stimulation. The amplitude of DBS therapy is often orders of magnitude greater than underlying neural signals causing the signal of interest to be heavily contaminated by artifact. In high resolution recordings, DBS artifact is typically removed using a simple low-pass filter. However, in low sampling rate recordings, the artifact is aliased into biologically significant frequency bands requiring more complex approaches to artifact removal. Stimulation artifacts may need to be removed from electrical stimulation recordings (e.g., LFP recordings) in order to identify neural biomarkers of disease symptoms and side effects of DBS. The period may be guessed by dividing the sampling rate by the stimulation frequency. Slight differences in external factors may make the sampling rate inexact. The true period may need to be found in order to produce accurate templates. A grid search may be done around the initial guess to find the best period. Periods may be evaluated via linear regression with a sum of sinusoidal harmonics of the period. The period which minimizes mean squared error with the raw data is chosen. Experimental results demonstrate that false periods can mimic harmonics of the artifact over short timespans. In some embodiments, to avoid such ‘distractor’ solutions, a penalty may be included for regression coefficients for high frequency sinusoids which are less significant for the true solution. After an optimal stimulation period is found, a region around each sample can be used to construct a local template. The value of the template corresponding to the phase of the sample can be subtracted in order to recover the artifact free recording.

Furthermore, it is expected that periodic artifacts in received waveform data from DBS and other electrical stimulations may cause multiple undesired and unknown phase shifts. Computationally efficient systems and methods are disclosed for period-based estimation of electrical stimulation artifacts in the presence of these multiple phase shifts.

Furthermore, as waveform data caused by deep brain stimulation is processed (e.g., to remove stimulation artifacts via PARRM), it can be expected that portions of the original waveform data may be lost in the transmission process. Various embodiments of the present disclosure describe novel and nonobvious systems and methods for estimating packet loss using the stimulation period (the systems and methods referred to herein as periodic estimation of lost packets (PELP)).

For example, during wireless transmission, neural data samples can be grouped into formatted units called ‘packets’. Packets contain a series of subsequent samples of a particular length as well as timing information and other relevant metadata. When transmitted, it is possible for packets to fail to reach the receiver leading to missing samples. These missing samples need to be properly accounted for when the time series is reconstructed. The timing information contained in each packet aids in this process but may be frequently inexact resulting in uncertainty in the number and location of the samples missing from a recording.

Particularly in less controlled environments away from the clinic, recordings can be especially prone to packet losses. Lower sampling rates may reduce the number of dropped packets and increase transmission ranges. However, it may still be typical for as much as 5% of the data to be lost even with such adjustments. Failure to accurately account for packet losses leads to the introduction of timing inaccuracies, artifacts during filtering, and reduced ability to identify meaningful neural signals.

Various embodiments for Periodic Estimation of Lost Packets (PELP) are disclosed herein, which include systems and methods for precisely estimating packet losses in bidirectional recordings from implanted devices where stimulation is active. PELP leverages a data driven procedure for determining the period of stimulation and the knowledge that stimulation continues identically during regions where data are missing to accurately account for the number of samples missing due to each dropped packet. As will be discussed herein, PELP may be robust across a range of amplitude ratios between stimulation and signal, pulse to pulse variations in stimulation amplitude, drift in stimulation frequency, and uncertainties in loss size estimates.

Streaming of intracranial electrophysiology data in the clinic and at home in ecologically valid environments may be essential for biomarker discovery in a variety of neurological disorders. Bidirectional implanted devices have enabled the acquisition of such datasets, however, data losses during wireless streaming hinder accurate analyses of neural signals. To address these issues, systems and methods for PELP are disclosed to precisely estimate and account for data losses from implanted recordings where stimulation is on.

Successful applications of PELP to recorded data are described herein that to using MEDTRONIC's SUMMIT RC+S from a human participant performing a behavioral task both in the clinic and at home to exactly estimate every occurrence of packet loss. PELP is widely applicable to other stimulating devices capable of wireless data streaming.

### Computer System

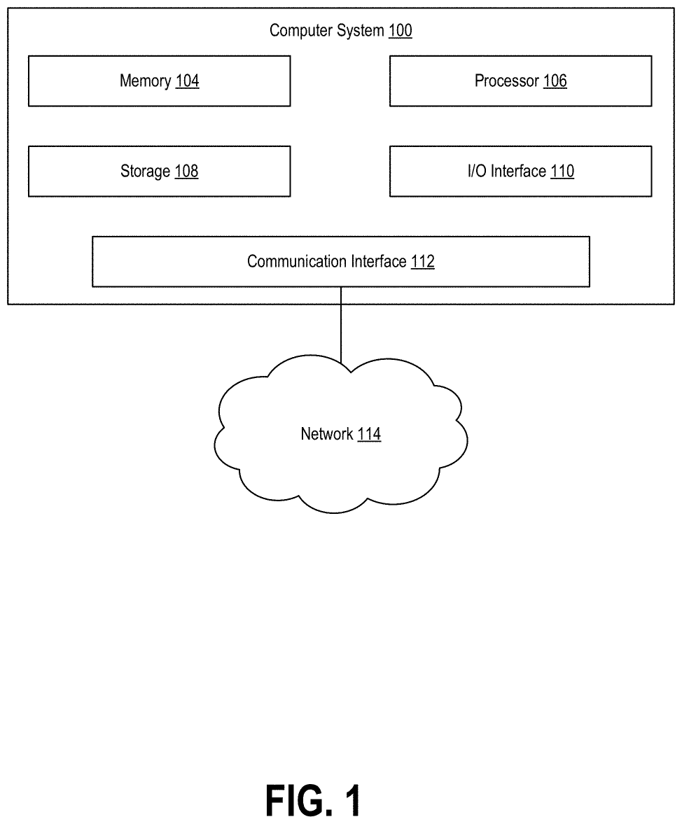

FIG. 1 illustrates an example computer system 100 that may be utilized to implement one or more of the devices and/or components discussed herein, including in the attached Appendix A. In particular embodiments, one or more computer systems 100 perform one or more steps or processes of one or more methods described or illustrated herein. In particular embodiments, one or more computer systems 100 provide the functionalities described or illustrated herein. In particular embodiments, software running on one or more computer systems 100 perform one or more steps or processes of one or more methods described or illustrated herein or provides the functionalities described or illustrated herein. Particular embodiments include one or more portions of one or more computer systems 100. Herein, a reference to a computer system may encompass a computing device, and vice versa, where appropriate. Moreover, a reference to a computer system may encompass one or more computer systems, where appropriate.

This disclosure contemplates any suitable number of computer systems 100. This disclosure contemplates the computer system 100 taking any suitable physical form. As example and not by way of limitation, the computer system 100 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, the computer system 100 may include one or more computer systems 100; be unitary or distributed; span multiple locations; span multiple machines; span multiple data centers; or reside in a cloud, which may include one or more cloud components in one or more networks. Where appropriate, one or more computer systems 100 may perform without substantial spatial or temporal limitation one or more steps or processes of one or more methods described or illustrated herein. As an example and not by way of limitation, one or more computer systems 100 may perform in real time or in batch mode one or more steps, processes, or blocks of one or more methods described or illustrated herein. One or more computer systems 100 may perform at different times or at different locations one or more steps, processes, or blocks of one or more methods described or illustrated herein, where appropriate.

In particular embodiments, computer system 100 includes a processor 106, memory 104, storage 108, an input/output (I/O) interface 110, and a communication interface 112. Although this disclosure describes and illustrates a particular computer system having a particular number of particular components in a particular arrangement, this disclosure contemplates any suitable computer system having any suitable number of any suitable components in any suitable arrangement.

In particular embodiments, the processor 106 includes hardware for executing instructions, such as those making up a computer program. As an example and not by way of limitation, to execute instructions, the processor 106 may retrieve (or fetch) the instructions from an internal register, an internal cache, memory 104, or storage 108; decode and execute the instructions; and then write one or more results to an internal register, internal cache, memory 104, or storage 108. In particular embodiments, the processor 106 may include one or more internal caches for data, instructions, or addresses. This disclosure contemplates the processor 106 including any suitable number of any suitable internal caches, where appropriate. As an example and not by way of limitation, the processor 106 may include one or more instruction caches, one or more data caches, and one or more translation lookaside buffers (TLBs). Instructions in the instruction caches may be copies of instructions in memory 104 or storage 108, and the instruction caches may speed up retrieval of those instructions by the processor 106. Data in the data caches may be copies of data in memory 104 or storage 108 that are to be operated on by computer instructions; the results of previous instructions executed by the processor 106 that are accessible to subsequent instructions or for writing to memory 104 or storage 108; or any other suitable data. The data caches may speed up read or write operations by the processor 106. The TLBs may speed up virtual-address translation for the processor 106. In particular embodiments, processor 106 may include one or more internal registers for data, instructions, or addresses. This disclosure contemplates the processor 106 including any suitable number of any suitable internal registers, where appropriate. Where appropriate, the processor 106 may include one or more arithmetic logic units (ALUs), be a multi-core processor, or include one or more processors 106. Although this disclosure describes and illustrates a particular processor, this disclosure contemplates any suitable processor.

In particular embodiments, the memory 104 includes main memory for storing instructions for the processor 106 to execute or data for processor 106 to operate on. As an example, and not by way of limitation, computer system 100 may load instructions from storage 108 or another source (such as another computer system 100) to the memory 104. The processor 106 may then load the instructions from the memory 104 to an internal register or internal cache. To execute the instructions, the processor 106 may retrieve the instructions from the internal register or internal cache and decode them. During or after execution of the instructions, the processor 106 may write one or more results (which may be intermediate or final results) to the internal register or internal cache. The processor 106 may then write one or more of those results to the memory 104. In particular embodiments, the processor 106 executes only instructions in one or more internal registers or internal caches or in memory 104 (as opposed to storage 108 or elsewhere) and operates only on data in one or more internal registers or internal caches or in memory 104 (as opposed to storage 108 or elsewhere). One or more memory buses (which may each include an address bus and a data bus) may couple the processor 106 to the memory 104. The bus may include one or more memory buses, as described in further detail below. In particular embodiments, one or more memory management units (MMUs) reside between the processor 106 and memory 104 and facilitate accesses to the memory 104 requested by the processor 106. In particular embodiments, the memory 104 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 104 may include one or more memories 104, where appropriate. Although this disclosure describes and illustrates particular memory implementations, this disclosure contemplates any suitable memory implementation.

In particular embodiments, the storage 108 includes mass storage for data or instructions. As an example and not by way of limitation, the storage 108 may include a hard disk drive (HDD), a floppy disk drive, flash memory, an optical disc, a magneto-optical disc, magnetic tape, or a Universal Serial Bus (USB) drive or a combination of two or more of these. The storage 108 may include removable or non-removable (or fixed) media, where appropriate. The storage 108 may be internal or external to computer system 100, where appropriate. In particular embodiments, the storage 108 is non-volatile, solid-state memory. In particular embodiments, the storage 108 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 108 taking any suitable physical form. The storage 108 may include one or more storage control units facilitating communication between processor 106 and storage 108, where appropriate. Where appropriate, the storage 108 may include one or more storages 108. Although this disclosure describes and illustrates particular storage, this disclosure contemplates any suitable storage.

In particular embodiments, the I/O Interface 110 includes hardware, software, or both, providing one or more interfaces for communication between computer system 100 and one or more I/O devices. The computer system 100 may include one or more of these I/O devices, where appropriate. One or more of these I/O devices may enable communication between a person (i.e., a user) and computer system 100. As an example and not by way of limitation, an I/O device may include a keyboard, keypad, microphone, monitor, screen, display panel, mouse, printer, scanner, speaker, still camera, stylus, tablet, touch screen, trackball, video camera, another suitable I/O device or a combination of two or more of these. An I/O device may include one or more sensors. Where appropriate, the I/O Interface 110 may include one or more device or software drivers enabling processor 106 to drive one or more of these I/O devices. The I/O interface 110 may include one or more I/O interfaces 110, where appropriate. Although this disclosure describes and illustrates a particular I/O interface, this disclosure contemplates any suitable I/O interface or combination of I/O interfaces.

In particular embodiments, communication interface 112 includes hardware, software, or both providing one or more interfaces for communication (such as, for example, packet-based communication) between computer system 100 and one or more other computer systems 100 or one or more networks 114. As an example and not by way of limitation, communication interface 112 may include a network interface controller (NIC) or network adapter for communicating with an Ethernet or any other wire-based network or a wireless NIC (WNIC) or wireless adapter for communicating with a wireless network, such as a Wi-Fi network. This disclosure contemplates any suitable network 114 and any suitable communication interface 112 for the network 114. As an example and not by way of limitation, the network 114 may include one or more of an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 100 may communicate with a wireless PAN (WPAN) (such as, for example, a Bluetooth® WPAN), a WI-FI network, a WI-MAX network, a cellular telephone network (such as, for example, a Global System for Mobile Communications (GSM) network), or any other suitable wireless network or a combination of two or more of these. Computer system 100 may include any suitable communication interface 112 for any of these networks, where appropriate. Communication interface 112 may include one or more communication interfaces 112, where appropriate. Although this disclosure describes and illustrates a particular communication interface implementations, this disclosure contemplates any suitable communication interface implementation.

The computer system 102 may also include a bus. The bus may include hardware, software, or both and may communicatively couple the components of the computer system 100 to each other. As an example and not by way of limitation, the bus may include an Accelerated Graphics Port (AGP) or any other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-PIN-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local bus (VLB), or another suitable bus or a combination of two or more of these buses. The bus may include one or more buses, where appropriate. Although this disclosure describes and illustrates a particular bus, this disclosure contemplates any suitable bus or interconnect.

### Period-Based Artifact Reconstruction and Removal for Deep Brain Stimulation

The development of closed-loop electrical neuromodulation therapies, for example adaptive Deep Brain Stimulation (aDBS) and adaptive Spinal Cord Stimulation (aSCS) can revolutionize the efficacy of neurostimulation therapies for treatment of many disorders, including Parkinson's Disease (PD), Epilepsy, Essential Tremor, Obsessive Compulsive Disorder (OCD), Treatment Resistant Depression (TRD), and chronic pain. While significant advances have been made in biomarker identification in PD and Epilepsy, there may not definitive biomarker for a single mental disorder, including OCD and TRD, or chronic pain. Biomarker identification and development of an adaptive neurostimulation system may require a hardware platform capable of simultaneous sensing and stimulation. This is particularly challenging when the neural signal of interest originates in or nearby the stimulation target as the amplitude of stimulation therapy is typically several orders of magnitude greater than the amplitude of signals of interest in the brain and spinal cord. Therefore, recordings for adaptive control may be heavily contaminated by high amplitude, high frequency stimulation artifact. In order to extract the underlying neural signatures of disease state, it may be necessary to remove the stimulation artifact.

Typically, high frequency artifacts are removed using a lowpass filter, however, limited sampling rates of existing implantable DBS and SCS devices and aliasing of stimulation pulses into low frequencies render lowpass filters ineffective. Stimulation artifact removal methods robust to aliasing may include: signal reconstruction via deletion and interpolation, decomposing and subtracting components of the signal related to the artifact, and subtracting a template of the artifact at each stimulation pulse. Deletion and interpolation may not be effective when artifact duration is long, and may not be ideal due to signal loss over the duration of each artifact. Signal decomposition methods have shown some success, but may require a large set of recording channels to be effective. While template subtraction methods have proven to be successful, such methods rely on accurate detection of each stimulation pulse. Existing methods for identifying individual stimulation pulses in recorded data (e.g. thresholding) are not robust to low sampling rates, the presence of other spurious high amplitude artifacts, or stimulation artifacts with broad peaks. Thus, there is a desire and need for a system and method that is effective at removing stimulation artifacts from local field potential (LFP) and other electrical stimulation recordings sampled at less than twice the frequency of stimulation without contaminating the underlying neural signal. The absence of such systems and methods thus greatly hinders control signal identification.

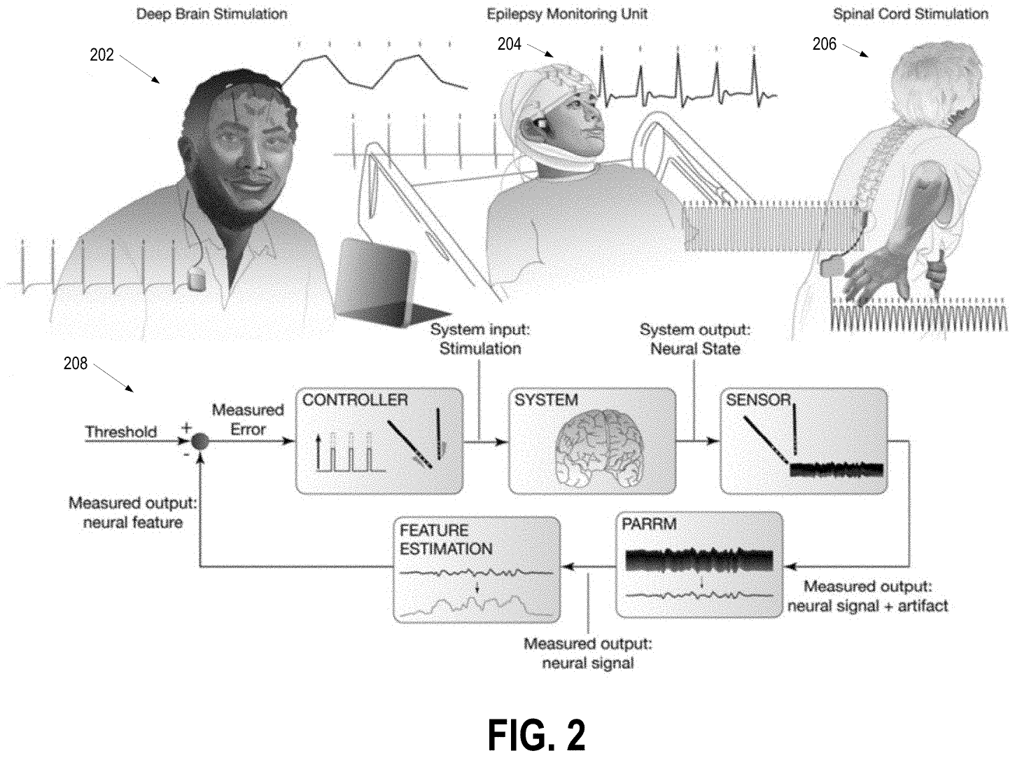

To overcome the challenges in removing periodic stimulation from neural recordings, various embodiments of the present disclosure describe a novel artifact removal method (e.g., a Period-Based Artifact Reconstruction and Removal Method (PARRM), to remove high frequency stimulation artifact in low and high-resolution LFP and other electrical stimulation recordings. As will be discussed herein, PARRM has superior performance to existing, state-of-the-art filters in saline experiments, computer simulations, and five unique in vivo recording paradigms. As will be discussed herein, PARMM can allow the recovery of a previously obscured biomarker in Parkinson's Disease participants and could be implemented online to perform real-time biomarker detection (e.g., as illustrated in FIG. 2).

FIG. 2 is a schematic illustrating the detection of artifacts during deep brain stimulation, according to a non-limiting embodiment of the present disclosure. PARRM may provide biomarker detection during ongoing neurostimulation (e.g., deep brain stimulation) to enhance efficacy of closed-loop neuromodulation. Deep brain stimulation may be applied at various frequencies via a medical device (e.g., ACTIVA PC+S, BLACKROCK CEREBUS, RIPPLE NOMAD, etc.) for the treatment of various medical condition. For example, deep brain stimulation may be applied at 150 Hertz to treat refractory obsessive compulsory disorders (OCD) (block 202), 120 Hz in an Epilepsy Monitoring Unit-like (EMU-like) scenario for treatment of treatment resistant depression (TRD) (block 204), and 50 Hz for treatment of chronic pain via spinal cord stimulation (SCS) (block 206). For each scenarios, as shown in FIG. 2, the blue traces show theoretical injected DBS waveform and black trace shows DBS waveform sampled in vivo at 200 Hz, 2 kHz, and 30 kHz, via a respective medical device used (e.g., ACTIVA PC+S, BLACKROCK CEREBUS, and RIPPLE NOMAD, respectively). FIG. 2 further shows a control policy 208 for the closed-loop DBS. In some embodiments, electrodes in the brain can sense neural signals and artifacts in real-time. Furthermore, the artifacts may be removed via PARRM, as discussed herein, without contaminating the underlying neural signal. The removal may allow feature estimation for the closed-loop control of stimulation amplitude to relieve symptoms.

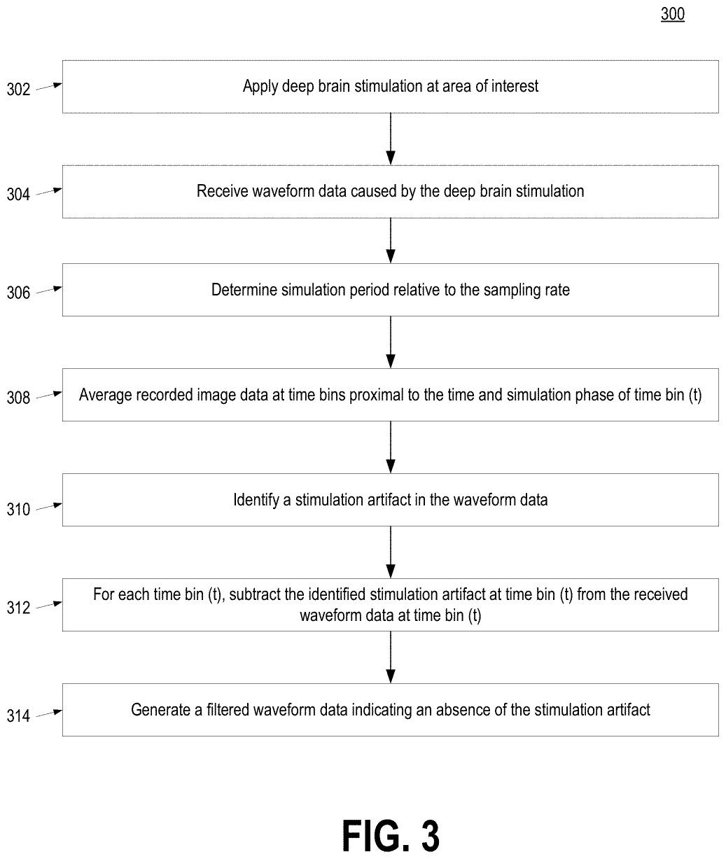

An example design of PARMM may involve subtracting an estimate of the stimulation artifact at each time bin from the recorded signal at that time bin. The artifact estimate may be formed by averaging the recorded signal at other time bins that are close to the current time bin in both time and stimulation phase. The artifact may bepresumed to be roughly identical for all of these time bins. Averaging may reduce the influence of brain signals and additional sources of noise, so that the estimate is primarily artifact. This process can be implemented as a linear filter (e.g., asaa weighted average using a sliding window). In some embodiments, PARRM may need a precise estimate of the stimulation period relative to the sampling rate. Slight inaccuracies in device system clocks can necessitate using a data-driven method to determine this period, which is done by finding the period that, when the data are divided into epochs the length of one period and overlapped, the samples will consolidate around the shape of the high-resolution artifact waveform. The complete process of data-driven period finding, artifact estimation, and signal reconstruction is illustrated in FIGS. 3-5.

FIG. 3 is a flow diagram showing an example method 300 of deep brain stimulation artifact identification and removal, according to a non-limiting embodiment of the present disclosure. One or more steps processes, or blocks of method 300 may be performed by computer system 100 (e.g., via its processor 106 based on instructions stored in memory 104). In some embodiments, the computer system 100 may comprise a computing device, and may be referred to as computing device 100.

Method 300 may begin with the application of a deep brain stimulation at a patient-specific area of interest (block 302). For example, as previously discussed in FIG. 2, Deep brain stimulation may be applied at various frequencies via a medical device (e.g., ACTIVA PC+S, BLACKROCK CEREBUS, RIPPLE NOMAD, etc.) for the treatment of various medical conditions.

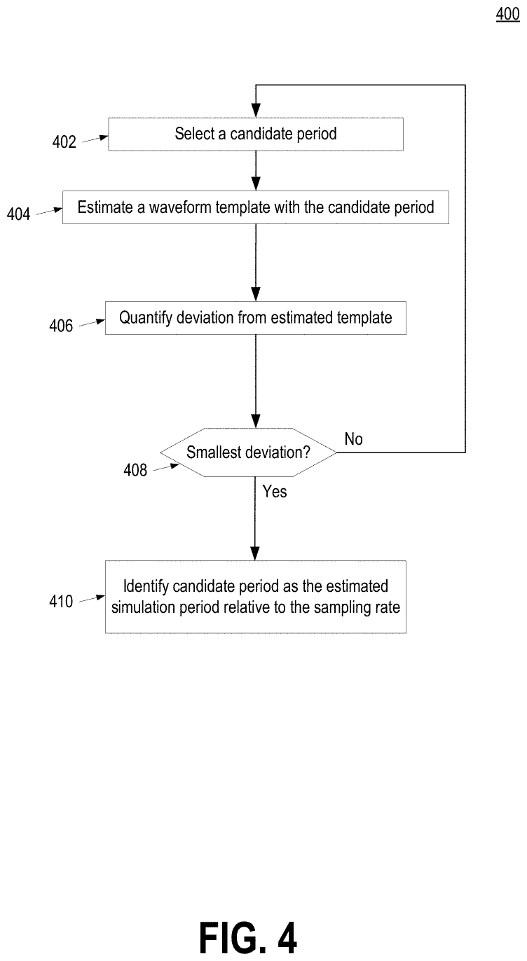

The computing device may thus receive waveform data causes by the deep brain stimulation (block 304). In some aspects, the waveform data may be received, and one or more subsequent steps, processes, or blocks may be performed, in real-time or near real-time. At block 306, the computing device may determine a stimulation period relative to a sampling rate. As will be discussed, method 400 shown in FIG. 4 describes examples of this block in more detail.

The computing device may identify, a stimulation artifact in the waveform data (block 310). In some aspects, the stimulation artifact may be identified by averaging recorded image data of the waveform data at time bins proximal to the time and/or proximal to the simulation phase of time bin (t) of the waveform data (block 308). The artifact may be presumed to be roughly identical for all of these time bins, including time bin t. Averaging may reduce the influence of brain signals and additional sources of noise, so that the subtracted signal may be deemed as primarily stimulation artifact. After the stimulation artifact is identified, the computing device may remove the stimulation artifact from the waveform data. Thus, for each time bin (t), the computing device may subtract the identified stimulation artifact at time bin (t) from the received waveform data at time bin (t) (e.g., the recorded signal at time bin (t)) (block 312).

For example, in some embodiments, blocks 308 through 314 may be shown through the following operations. Let T denote the stimulation period relative to the sampling rate (in units of sampling time bins) (e.g., as determined in block 306). The time bins included in the average (e.g., determined in block 308) may be those times bins s such that Nₛₖᵢₚ<|s−t|≤N_{bins} and such that |s−t| (mod T)≤D_{period} or |s−t| (mod T)≤T−d_{period}, where α (mod T) denotes α modulo T, and where 0≤Nₛₖᵢₚ<N_{bins} and 0≤D_{period}≤T are user-chosen design parameters. In some aspects, the additional criterion s−t<0 can be included so that only past observations are used to estimate the stimulation artifact. Let Bₜ denote the collection of those times bins s that are used for averaging and let |Bₜ| denote the number of such time bins. Using rₜ to denote the recorded signal at time bin t, the corrected signal is cₜ defined by

$${c}_{t} = {r}_{t} - \frac{1}{\vert {B}_{t} \vert} {\sum }_{s ∈ {B}_{t}} {r}_{s} = {\sum }_{i = - {N}_{bins}}^{{N}_{bins}} {W}_{i} {r}_{t - i} ,$$

where wᵢ is a list of weights defined by w₀=1, and wᵢ=−1/B₀| if −i ϵ B₀, and wᵢ=0 otherwise. The final expression shows that the PARRM correction can be implemented by a fixed linear filter (with the filter weights denoted by w′), making it fast and simple to implement. (e.g., if the additional criterion s−t<0 is used, then the final summation would begin at i=0. The final methodology may be modified near the start and end of stimulation.

In some embodiments, the design parameters for the PARRM filter are N_{bins}, Nₛₖᵢₚ, and D_{period}. Larger choices of Nbins allow more data to be averaged in order to estimate the artifact, reducing estimation variability. But, larger choices of Nbins also lengthen the temporal window used to estimate the artifact, perhaps introducing estimation bias if the artifact shape is changing in time. Because neural signals have temporal autocorrelation, it is important to avoid averaging data too close to time bin t or the neural signal itself could be subtracted during artifact removal. Larger choices of Nₛₖᵢₚ help to mitigate this danger, but also reduce the amount of data used to estimate the artifact. Similar to N_{bins}, larger choices of D_{period} allow more data to be averaged, but also introduce more estimation bias by temporally smoothing the estimated artifact. The optimal choices for these design parameters may vary depending on the situation.

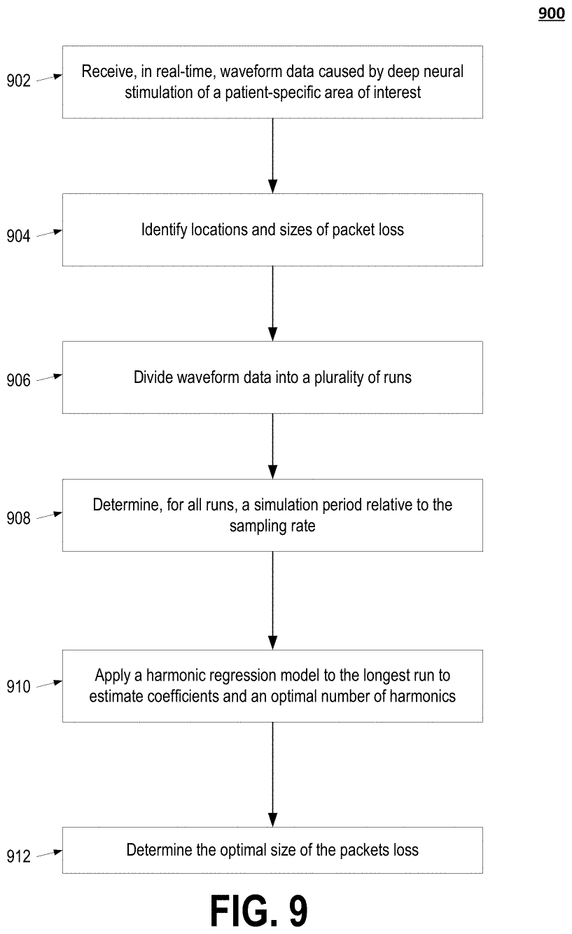

FIG. 4 is a flow diagram showing an example method 400 of determining a stimulation period, T, relative to a sampling rate, according to a non-limiting embodiment of the present disclosure. PARRM may need a precise estimate T of the stimulation period relative to the sampling rate. T can be determined via several methods, such a method 400. Moreover, method 400 may include an example process for performing block 306 of method 300 (previously discussed and shown in FIG. 3). Also, as will be described further below, method 400 may include an example process for performing block 908 of method 900 (shown in FIG. 9). One or more steps, processes, or blocks of method 400 may be performed by computer system 100 (e.g., via its processor 106 based on instructions stored in memory 104). In some embodiments, the computer system 100 may comprise a computing device, and may be referred to as computing device 100.

Method 400 is an automated, data-driven method to determine the simulation period, T, by searching for a period that creates a strongly resolved template. Method 400 begins with selecting a candidate period, δ>0 (block 402). For each candidate period δ>0, the method estimates a waveform template with this period (block 404). The computing device may thus quantify a deviation from the estimated template (block 406). For example, for each candidate period, f_{B,δ}where the potential period is δ>0, and β=(β=, . . . , β>₂ₘ₊₁) is a parameter vector, the computing device may determine whether a deviation between the candidate period, f_{B,δ}and an estimated waveform template (e.g., its amplitude, yₖ) is the smallest (block 408). If not the smallest, another candidate period may be selected. The candidate period with the smallest deviation may be identified as the final estimate T of the period that is used by PARRM (block 410).

In at least one embodiment, the aforementioned blocks may be described by the following operations. Let m≥0 be an integer. For each potential period δ>0 and each parameter vector β=(β=, . . . , β>₂ₘ₊₁) define the functions

$${f}_{B , δ} ( t ) = {B}_{1} + {\sum }_{j = 1}^{m} {B}_{2 j} sin ( \frac{2 π jt}{δ} ) + {\sum }_{j = 1}^{m} {B}_{2 j + 1} cos ( \frac{2 π jt}{δ} )$$

The function f_{B,δ}is a periodic function with period δ. Each f_{B,δ}is a candidate artifact waveform. The parameter vector β controls the strength of the different frequencies that define f_{B,δ}, and m controls the number of allowed frequencies. Let ((tₖ, yₖ): k=1, . . . , n) be a collection of (time, value) pairs. The yₖ value used here is the change in recorded electrical stimulation (e.g., LFP) amplitude at time tₖ with some preprocessing to obtain standardized units, reduce the influence of outliers, and reduce the size of the dataset. Mean squared error is used to measure how well the function f_{B,δ}, fits these pairs:

$$mse = ( β , δ ) = \frac{1}{n} {\sum }_{k = 1}^{n} {( {y}_{k} - {f}_{β , δ} ( {t}_{k} ) )}^{2} .$$

In some embodiments, the minimization over δ may complicated by many local minima, spurious ‘distractor’ solutions that mimic the harmonics of the true waveform, and high sensitivity to small changes in δ. In some aspects, a penalized, stagewise search that begins with smaller intervals of data (to reduce the sensitivity to δ), smaller m (to reduce the number of local minima), and a penalty for higher frequency solutions (to help avoid distractor solutions) may be used to overcome these issues. After the stimulation period, T is found, it may be fixed for PARRM (e.g., as previously discussed in method 300). Furthermore, as will be discussed in FIG. 9, the stimulation period, T, may also be used for methods to estimate packet loss from waveform data caused by deep brain stimulation.

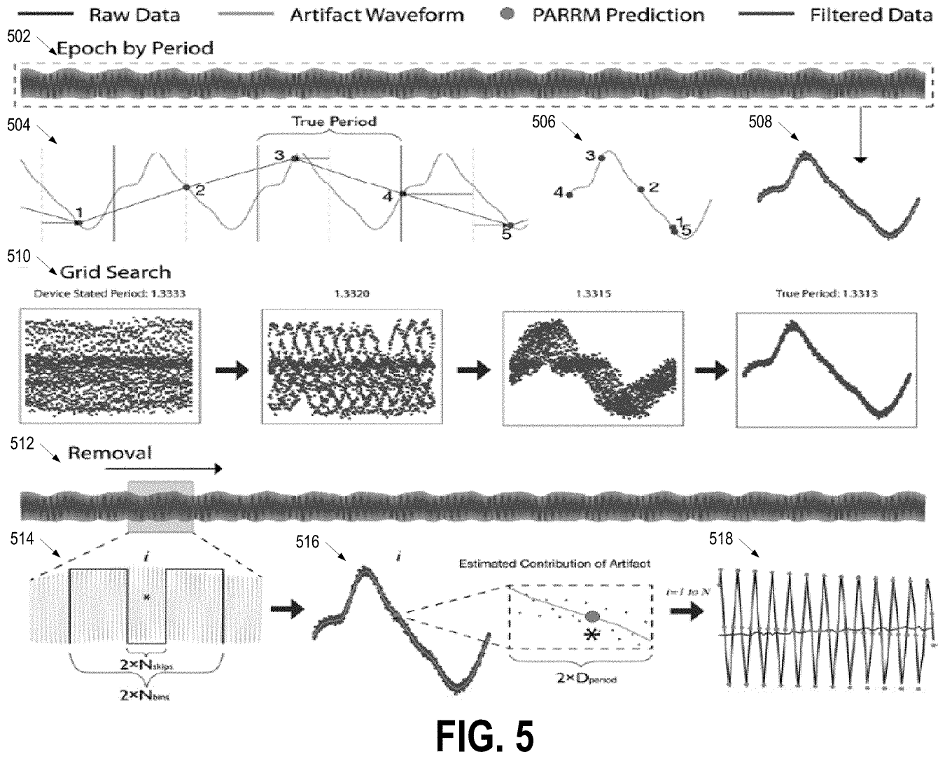

FIG. 5 is a schematic illustrating stimulation period determination, stimulation artifact reconstruction, and stimulation artifact removal, according to a non-limiting embodiment of the present disclosure. For example, graph 502 shows an example LFP recording sampled at 200 Hz. This LFP recording may be used to identify the true stimulation period (e.g., via previously discussed method 400). Furthermore, graph 504 shows an illustration of a 5-sample snippet of the LFP recording divided into epochs using the true stimulation period and overlaid with a high-resolution waveform (light blue). The black points in graph 504 indicate individual raw LFP samples. In graph 506, the epochs for all 5 samples are overlaid on the timescale of the true stimulation period. As shown in graph 508, when all epochs in the recording are overlaid using this procedure, all samples consolidate around the shape of the high-resolution artifact waveform on the timescale of the true stimulation period. As shown in graph 510, the period suggested by the device sampling and stimulation rates is inexact and may not result in a consolidated waveform. Thus, using a grid search centered around the stated period, a series of periods may be evaluated to find the true period that produces the most consolidated samples. As shown in graph 512, a sliding window can be applied to the entire recording to estimate the contribution of the stimulation artifact at each sample. Graph 514 shows how, for each window, a rectangular kernel (length N_{bins}), ignoring the center (length Nₛₖᵢₚₛ) can be used to estimate the value of the artifact at each sample of interest i (asterisk). Furthermore, as shown in graph 516, samples within a distance, D_{period}, on the timescale of the artifact period can be averaged together to produce the estimate of the amplitude of the artifact (orange point) at sample i. As shown in graph 518, the estimated value of the artifact can then subtracted at each sample over the entire recording to recover the signal of interest (dark blue).

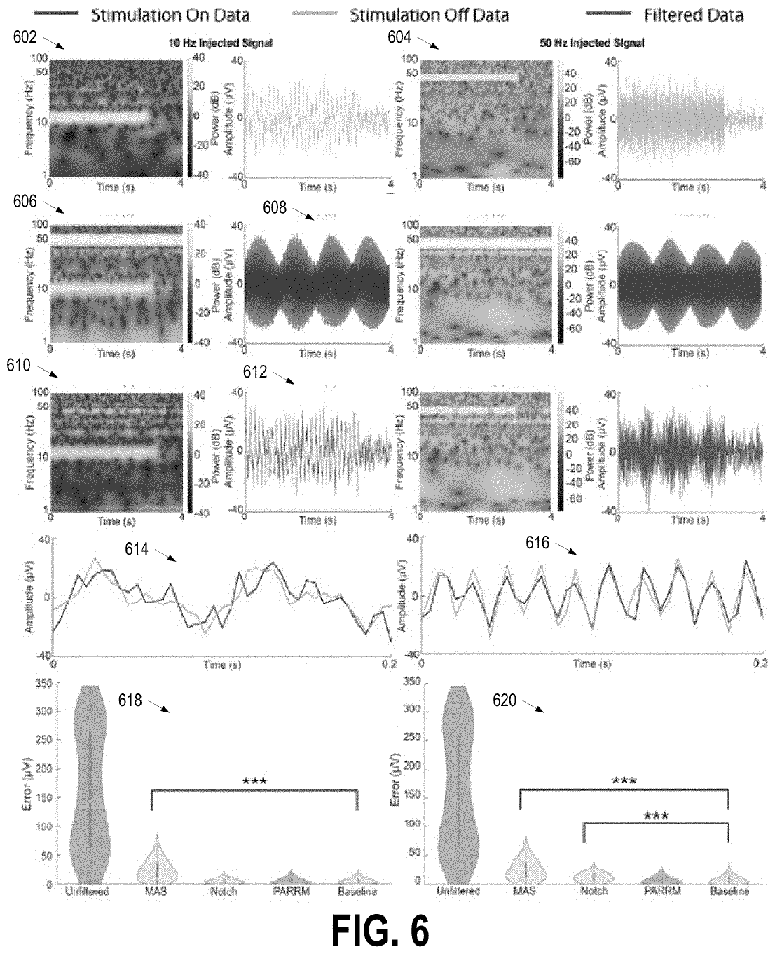

FIG. 6 shows example spectrograms and charts illustrating the recovery of sinusoidal signals during deep brain stimulation artifact identification and removal, according to a non-limiting embodiment of the present disclosure. PARRM may be used to remove the DBS artifact and recover the underlying injected signal and noise in saline. In artifact free (e.g., DBS off) recordings, both the 10 and 50 Hz injected signals can be clearly visible both in the frequency and time domains prior to signal offset (e.g., as shown in graphs 602 and 604). When stimulation is turned on, high amplitude artifacts may be visible in the frequency domain at 0 and 50 Hz, obscuring the 50 Hz injected signal but not the 10 Hz signal. In the time domain, both the 10 Hz and 50 Hz signals may be obscured (e.g., as shown in graphs 606 and 608). After filtering using PARRM, the effects of stimulation can be removed in both the frequency and time domains (e.g., as shown in graphs 610 and 612). In the case of the 50 Hz signal, this can achieved despite the artifact being aliased to the same frequency as the injected signal.

The similarity between the artifact free and filtered signals is visually apparent in 0.2 seconds of data for both the 10 and 50 Hz injected signals (e.g., as shown in graphs 614 and 616). Afterwards, filter performance can be quantified by comparing the distribution of absolute errors between artifact free signals and unfiltered, MAS filtered, notch filtered, and PARRM filtered signals to a baseline noise recording (e.g., no stimulation and no injected signal) (e.g., as shown in graphs 618 and 620). Filtering using MAS may not reduce the error to the level of baseline for either injected signal (p<0.0005). While effective at 10 Hz (p>0.05), notch filtering removed the injected signal along with the artifact leading to a large reduction in error yet still significantly different from baseline (p<0.0005). For both the 10 and 50 Hz injected signals, PARRM outperformed the other methods with no significant difference (p>0.05) from baseline, indicating that the remaining errors were expected due to noise in saline.

### Comparison of PARRM to Conventional Filters

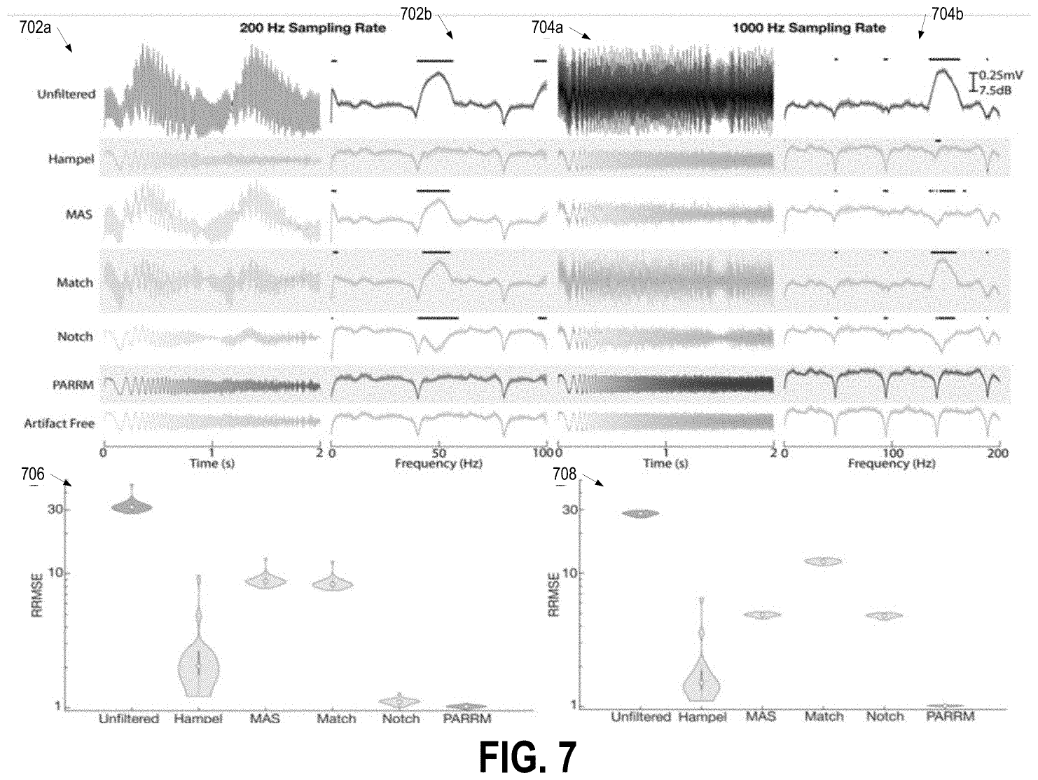

Having shown that PARRM is effective for recovering simple sinusoidal signals recorded in saline, a comparison of the method's performance to conventional filters in recovering more complex, injected, chirp signals for simulated data is discussed. FIG. 7 shows computer simulations comparing the deep brain stimulation artifact identification and removal to conventional filters, according to a non-limiting embodiment of the present disclosure. For example, graphs 702a and 702b show averaged time-voltage series 702a and windowed power spectral density 702b of 30 simulated linear chirps (0 Hz to 100 Hz, 2 second duration, variable separation) during concurrent 150 Hz stimulation for unfiltered, Hampel filtered, MAS filtered, Match filtered, Notch filtered, PARRM filtered, and artifact free recordings sampled at 200 Hz. The black solid bars indicate significant difference from artifact free signal (2-Sample t-test, p <0.05). When all chirps were averaged, PARRM recovered a signal with minimal distortion and noise in the time domain at both low and high sampling rates, unlike MAS and matched filters (e.g., as shown in graphs 702a and 704a). Additionally, graphs 702a and 702b show average time-voltage series 702a and average windowed power spectral density 702b of 30 simulated linear chirps (0 Hz to 200 Hz, 2 second duration, variable separation) during concurrent 150 Hz stimulation for unfiltered, Hampel filtered, MAS filtered, Match filtered, Notch filtered, PARRM filtered, and artifact free recordings sampled at 1000 Hz. The black solid bars indicate significant difference from artifact free signal (2-Sample t-test, p<0.05). PARRM showed no significant differences in the frequency domain at either sampling rate (e.g., as shown in graphs 702b and 704b). This was true even at frequencies affected by artifact where other filters either over (notch) or under (MAS and matched) filtered. Lastly, graphs 706 and 708 show an evaluation of filter performance based on time domain relative root mean squared error (RRMSE) (e.g., a ratio between mean squared error of artifact free versus theoretical chirp to the mean squared error of filtered versus theoretical chirp) of simulated chirps during concurrent 150 Hz stimulation sampled at 200 Hz and 1000 Hz. PARRM had a relative root mean squared error (RRMSE) close to one for both sampling rates indicating effective signal recovery on a single trial basis exceeding performance of the Hampel filter (e.g., as shown in graphs 706 and 708). For all three metrics, PARRM exceeded performance of all other filters for both low and high sampling rates.

Next, a parameter sweep was performed to test the effect of varying chirp length (1-10 s), amplitude (0.5-5 V), pulse width (30-180 μV), and frequency (80-180 Hz) on PARRM performance, measured by RRMSE and Relative R Ratio (S. FIG. 5). Effects for chirp length and pulse width were all within the margin of error. RRMSE and Relative R Ratio increased for increasing stimulation amplitude. RRMSE and Relative R Ratio decreased for stimulation frequencies above 100 Hz. All changes were at most 8% different from baseline indicating that PARRM performed well for a wide range of stimulation parameters and recorded signals. PARRM significantly attenuates stimulation artifacts from the ACTIVA PC+S device.

### Periodic Estimation of Lost Packets From Deep Brain Stimulation Waveform Data

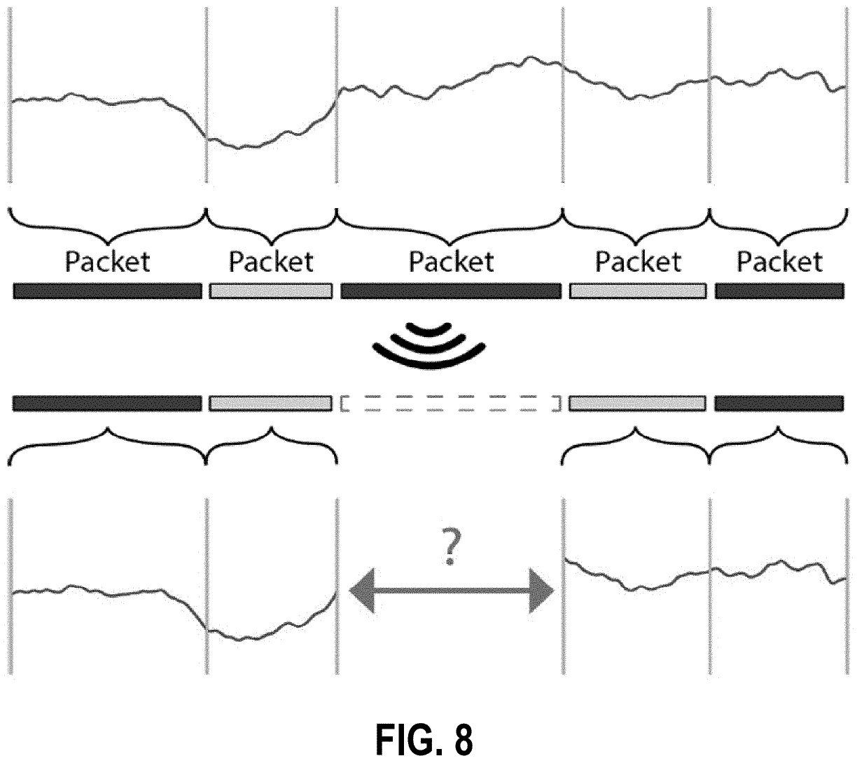

As waveform data caused by deep brain stimulation is received by the computing device 100, it can be expected that portions of the original waveform data (e.g., packets) may be lost in the transmission process. For example, FIG. 8 is a schematic illustrating packet loss in waveform data from deep brain stimulation, according to a non-limiting embodiment of the present disclosure. As implanted devices used for deep brain stimulation and acquire neural data for transmission (e.g., waveforms) over time, each subsequent sample of the neural data acquired (e.g., as part of a time series) by the implanted device are grouped into packets. These packets can then be wirelessly transmitted to a receiver (e.g., computing device 100). During the transmission process, it may be possible for some packets to be lost. As a result, the relative timing of the samples contained in received packets may be uncertain. Various embodiments of the present disclosure are disclosed for estimating such packet loss. Novel and nonobvious systems and methods are thus disclosed for estimating a quantity of samples missing due to a packet loss for recordings where stimulation is present, the systems or methods referred to herein as Periodic Estimation of Lost Packets (PELP).

For example, FIG. 9 is a flow diagram showing an example method 900 of a period-based estimation of packet loss (also referred to herein as Periodic Estimation of Lost Packets (PELP)), according to a non-limiting embodiment of the present disclosure. One or more steps, processes, or blocks of method 900 may be performed by computer system 100 (e.g., via its processor 106 based on instructions stored in memory 104). In some embodiments, the computer system 100 may comprise a computing device, and may be referred to as computing device 100. Method 900 may begin with the computing device receiving, in real-time, waveform data caused by deep neural stimulation of a patient-specific area of interest (block 902). For example, the patient-specific area of interest may comprise an area or section of a brain of a patient being treated, on which electrodes may be applied.

In some embodiments, before subsequent steps, processes, or blocks of PELP can be applied to the received recording of waveform data, the locations of packet losses and their sizes may initially be determined (block 904). In some aspects (e.g., in recordings by the SUMMIT RC+S device), each packet may have has three integer timing variables of note for this purpose: (1) ‘dataTypeSequence’, which may indicate the packet number that rolls over every 256 packets, (2) ‘systemTick’ time of the last sample in a packet with 0.1 ms resolution that rolls over every 6.5536 s, and (3) ‘timestamp’ with 1 s resolution and no rollover. A packet loss may occur when the dataTypeSequence between subsequent packets skips an index or the timestamps is more than 6 seconds apart corresponding to cases of large losses where the dataTypeSequence may roll over. For sampling rate fs, the number of samples lost, N, can be estimated according to the following equation when there is no systemTick rollover: N=((s₂−s₁−10⁴ fₛ⁻¹ (n−1)) mod 65536)×10⁻⁴, here s1 and s2 are the system ticks of the packets preceding and following the loss respectively and n is the number of samples in the packet after the loss. If systemTick rollover occurs, estimation may no longer be acceptably accurate due to timing drift between the systemTick and timestamp, and may need to be reset using a coarser metric, such as the UNIX timestamp (±50 ms vs. ±3 ms) corresponding to the time when the packet was received or generated. These estimates may not be sufficiently accurate to ensure the exact reconstruction of the timing between received packets down to sample resolution. PELP leverages the presence of regular stimulation in both the received and missing data to ensure precise estimates of data losses.

PELP may proceed by dividing the time series into a set of R continuous ‘runs’ (block 906). Each run may comprise a continuguous packet that is separated from another run by losses (block 906). Thus, computing device 100 may recognize runs (e.g., based on non-contiguos nature of the received waveform) and identify such runs accordingly.

At block 908, the computing device may determine, for each run, a period of stimulation (6) (e.g., from the waveform data of each respective run) relative to the sampling rate. The computing device 100 may use the period estimation component of PARRM, as previously discussed (e.g., block 304 of FIG. 3, method 400 of FIG. 4).

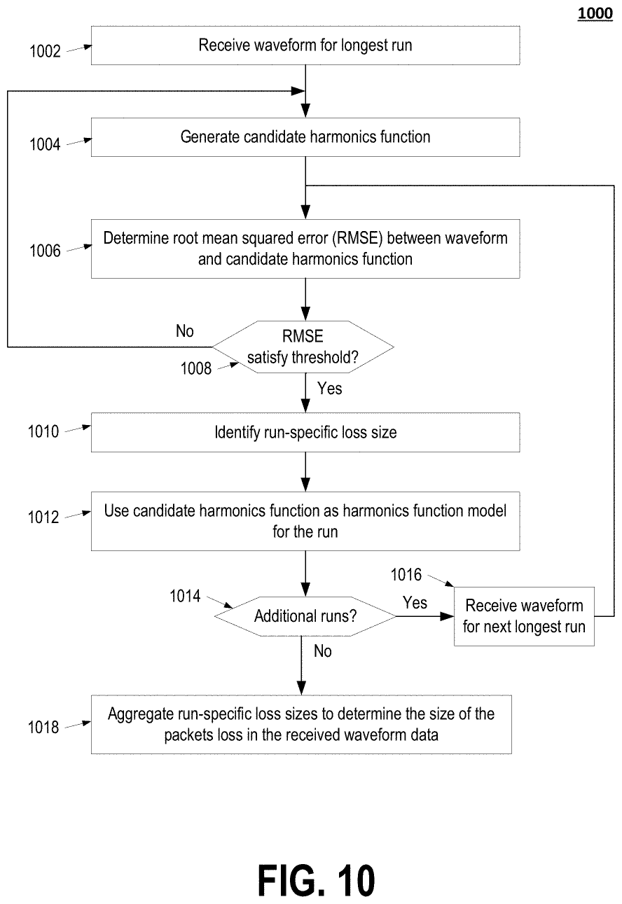

A harmonic regression model, f_{β,δ}, is then fit to the longest run to estimate the coefficients (β) and the optimal number of harmonics (m) (block 910). The optimal number of harmonics (m) may be chosen using Akaike Information Criterion (AIC). In some embodiments, additional harmonic regression models may be fit, e.g., for other runs of the received waveform data. The computing device 100 may thus use the harmonic regression model, f_{β,δ}, to determine the optimal size of the packets loss in the received waveform data (block 912). An example process for performing blocks 910 and 912 (e.g., training and applying a harmonic regression model and determining the optimal size of packet loss) is explained in more detail in method 1000 shown in FIG. 10.

FIG. 10 is a flow diagram showing an example method 1000 of applying a harmonic regression model to estimate packet loss, according to a non-limiting embodiment of the present disclosure. One or more steps, processes, or blocks of method 1000 may be performed by computer system 100 (e.g., via its processor 106 based on instructions stored in memory 104). In some embodiments, the computer system 100 may comprise a computing device, and may be referred to as computing device 100.

Method 1000 may begin with the computing device receiving a plurality of runs, including the longest run (block 1002), of the received waveform from the deep brain stimulation of method 900. At block 1004, the computing device may generate a candidate harmonics function, f_{B,δ}(t) for the training process. An example candidate harmonics function, f_{B,δ}(t), and the variables and coefficients to be determined for the candidate harmonics function is explained below:

$${f}_{B , δ} ( t ) = {B}_{1} + {\sum }_{j = 1}^{m} {B}_{2 j} sin ( \frac{2 π jt}{δ} ) + {\sum }_{j = 1}^{m} {B}_{2 j + 1} cos ( \frac{2 π jt}{δ} )$$

Let ((t_{k,r,}y_{k,r}): k=1, . . . , nᵣ: r=1, . . . , R) be a collection of (time, value) pairs.

At block 1006, the computing device may determine a root mean squared error (RMSE) between the waveform corresponding to the run (e.g., the longest run) and a candidate harmonics function. In some aspects, the waveform corresponding to the run may be represented by a function that outputs the waveform's amplitude. For example, a value, y_{k,r} may be used to represent the change in recorded electrical stimulation (e.g., LFP) amplitude of a run, r, at a time relative to the start of the recording t_{k,r} for run r. Root mean squared error (RMSE) may be used to measure how well the candidate harmonics function f_{B,δ}(t) fits these pairs for a packet loss size Δ:

$$RMSE ( Δ , r ) = \sqrt{\frac{1}{{n}_{r}} {\sum }_{k = 1}^{{n}_{r}} {( {y}_{k , r} - {f}_{β , δ} ( {t}_{k , r} - Δ ) )}^{2}}$$

Since, an objective may be to find a candidate harmonics function for which the RMSE between the candidate harmonics function and the waveform is at a minimum, t the optimal size of the packets loss for run r, Δᵣ, may therefore be: Δᵣ=argmin_{Δ}(rmse(Δ,r)).

As different candidate harmonic functions are tested, the computing device may determine, at each iteration, whether a given harmonic function satisfies a threshold (block 1008). For example, the threshold may be based on whether a previously held record of a minimum RMSE has been beaten.

After an RMSE satisfies a threshold (e.g., a minimum RMSE is reached between a candidate harmonics function and the waveform corresponding to the run), the computing device 100 may identify, at block 1010 the loss size associated with the run (run-specific loss size). Furthermore, the computing device may use the candidate harmonics function (for which the RMSE satisfied the threshold) as the harmonics function model for the run (e.g., at block 1012).

Blocks 1002 through 1012 were thus explained for determining a harmonics function model based on the longest run. The harmonics model may be applied to other runs of the waveform data received in method 900. In some embodiments, other harmonics model may be determined for other runs of the waveform data. Thus, if the computing device determines that there are additional runs (block 1014—Yes), the computing device 100 may receive, at block 1016, the waveform for the longest run of the additional runs (i.e., the next longest run of the entire waveform received in method 300). Blocks 1006 through 1014 may thus be repeated for each next longest run until all runs of the entire waveform data has been assessed. At each iteration, the run-specific loss sizes may be identified and stored.

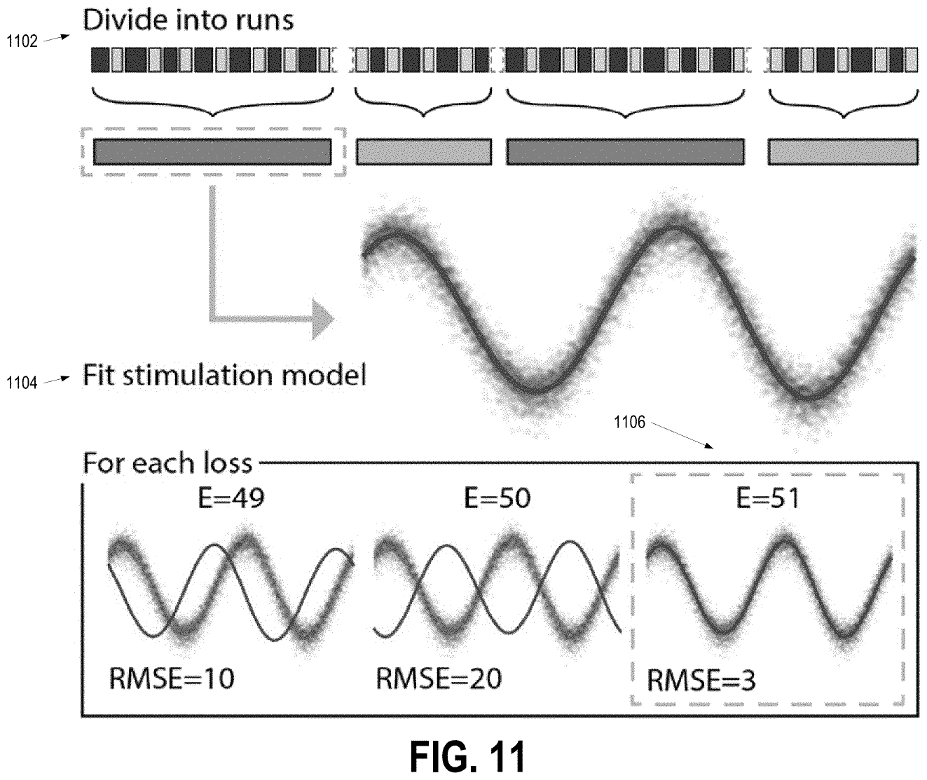

At block 1018, the computing device 100 may aggregate run-specific loss sizes (e.g., identified at block 1010 during one or more iterations) to determine the size of the packets loss in the received waveform data. The method of estimating packet loss is further illustrated in FIG. 11.

FIG. 11 is a schematic illustrating a period-based estimation of packet loss (PELP), according to a non-limiting embodiment of the present disclosure. As shown in marker 1102, PELP begins by grouping contiguous packets into continuous runs where each run is separated from adjacent runs by losses. However, loss sizes are estimated but may be uncertain. The stimulation period may be analytically determined using the entirety of the waveform data (e.g., as previously explained in FIGS. 3 and 4). As further shown via marker 1104, a stimulation model (e.g., a harmonic function) may be fit to the longest run via harmonic regression. For all other runs, the root mean squared error (RMSE) between the model and the samples in the run is computed for a range of loss sizes centered around the estimate. As shown in box 1106, the loss size that minimizes the RMSE can be selected as the true loss size.

### Experimental Testing of the Period-Based Estimation of the Loss of Packets (PELP)

Various experiments were performed using waveforms received from deep brain stimulation that show the effectiveness of PELP as a method of determining packet loss. In at least one experiment, neural data was recorded from one participant undergoing DBS surgery for treatment of obsessive compulsive disorder (OCD). Electroencephalography (EEG) data were recorded both with and without stimulation when the participant visited the clinic for DBS programming. LFP data were recorded both in the clinic and when the patient was at home. Electrodes on both the right and left sides were implanted in the VC/VS according to standard stereotactic procedures using computed tomography for target determination. The location of electrode placement was made entirely on clinical grounds. Bilateral 150.6 Hz stimulation with a pulse width of 90 ps and amplitude of 4 mA for the left side and 4.5 mA for the right side was used for all recordings where stimulation was turned on. The participant gave informed consent and data presented were collected in accordance with recommendations of the federal human subjects regulations and under protocol H-40255 approved by the Baylor College of Medicine Institutional Review Board.

Continuous electroencephalography (EEG) was recorded using a 64-channel ACTICAP BRAIN VISION SYSTEM (BRAIN VISION, Morrisville, N.C., USA). A common mode sense electrode was located at FCz. The EEG was band-pass filtered online between 0.1 and 1000 Hz and digitized at 5 kHz. The EEG was downsampled offline to 1000 Hz with an anti-aliasing filter prior to analysis. The continuous LFP was recorded using the SUMMIT RC+S (MEDTRONIC, Minneapolis, Minn., USA) via wireless data streaming from implanted electrodes to the device running the task. Each DBS probe (Model 3387, one per hemisphere) included four electrode contacts two of which were used per side to conduct bipolar recordings. LFP recordings were sampled at 1 kHz in the clinic and 250 Hz at home in order to minimize data losses. Signal processing and analysis were performed in MATLAB (MATHWORKS, Natick, Mass., USA) using in-house code.

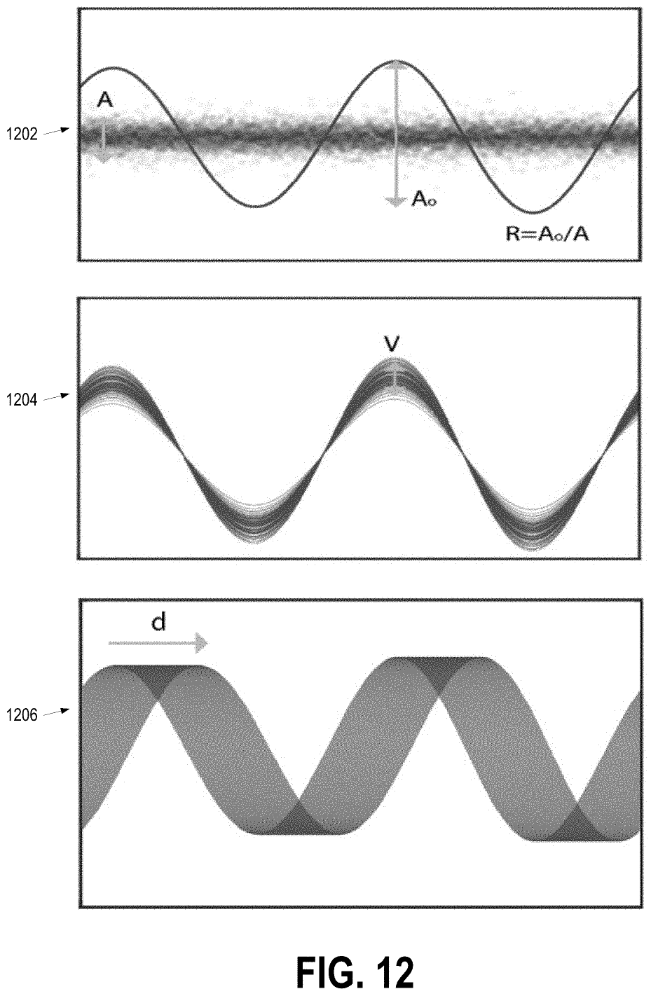

In order to simulate stimulation in the DBS recordings, DBS artifacts were modeled as a sum of sinusoidal harmonics of the stimulation frequency (e.g., as previously discussed in FIGS. 9 and 10). The effect of stimulation was simulated by adding the artifact component regressed from recordings on a different day where stimulation was turned on to data without stimulation. A high-pass filter at 1 Hz with a Gaussian window was first applied to achieve approximately 40 dB attenuation in the stopband before the period of stimulation (δ) was identified using the period estimation component of PARRM. A sum of sinusoids f_{β,δ}(t) with m harmonics of the period and coefficients β was then fit to the data using linear regression. The stimulation amplitude for each cycle was sampled from a normal distribution with mean A1 and standard deviation v. The mean stimulation amplitude was set relative to the root mean squared amplitude of the stimulation off data (A) according to a ratio (R) and the original root mean squared amplitude of the fit (Ao). In order to model potential inaccuracies in period estimation, the period of the stimulation model was slightly offset by a drift factor (d) measured in percent drift per 1000 cycles from the period used during PELP. The effects of these three parameters are illustrated in FIG. 12.

FIG. 12 shows sinograms illustrating the fitting of a stimulation model to waveform data, according to a non-limiting embodiment of the present disclosure. As shown in sinogram 1202, the root mean squared amplitude (Ao) of the stimulation model is set relative to that of the neural signal of interest (A) according to a target ratio R. In sonogram 1204, the amplitude of each stimulation pulse is varied on a cycle-wise basis where the amplitude of each pulse is sampled from a normal distribution with mean Ao and standard deviation V. Furthermore, as shown in sinogram 1206, inaccuracies in period estimation, drifting sampling rate, and frequency variability are modeled by adding a drift factor d to the stimulation period in the model.

Furthermore, three sets of experiments were performed to simulate the accuracy of loss estimation while varying different parameters in the stimulation model. For each experiment, Monte Carlo analyses were used to simulate a large number of experiments by randomly sampling subsets of 50 sample “packets” to remove from the recording. These simulations were applied while varying one of amplitude ratio, amplitude variability, or drift as a function of the loss uncertainty. For each simulation, the uncertainty ranged from 0-50 samples in 1 sample increments while the dependent parameters ranged from 0-4 in increments of 0.1 for the amplitude ratio, 0-10% in increments of 1% for the amplitude variability, and 0-0.6% in increments of 0.015% per 1000 cycles for the drift. PELP was applied to each simulation to determine the proportion of losses it was able to estimate correctly depending on the stimulation parameters.

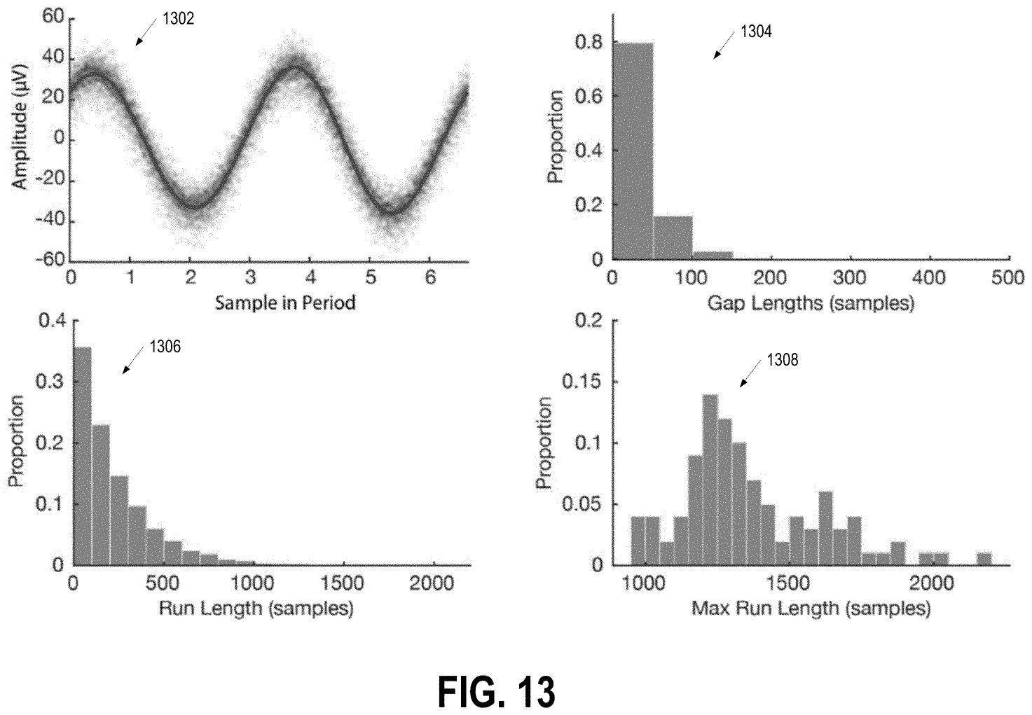

FIG. 13 shows graphs illustrating results of the stimulation model in the above described experiments, according to a non-limiting embodiment of the present disclosure. For example, the top left panel 1302 shows the stimulation model compared to the raw EEG samples overlapped by computing the modulo of each timepoint with the model's period of stimulation. The period of stimulation was found to be 6.64000 samples. Four sinusoidal harmonics were used for the fit based on model selection via AIC. Raw samples are well consolidated about the artifact waveform with a residual standard deviation of 5.90 μV similar to the standard deviation of the stimulation off data (4.55 μV).

The histograms 1304-1308 in FIG. 4 illustrate features of the Monte Carlo simulation of 100 loss experiments. As shown in histogram 1304, the average length of a missing data gap was 63 samples with a median of 50 samples (one loss). As shown in histogram 1306, runs of continuous samples between losses ranged from 50-2200 samples with an average length of 251 samples. As shown in histogram 1308, the max run length in each simulation ranged from 950-2200 samples with an average length of 1342 samples corresponding to roughly 202 cycles of the 150.6 Hz simulated stimulation frequency.

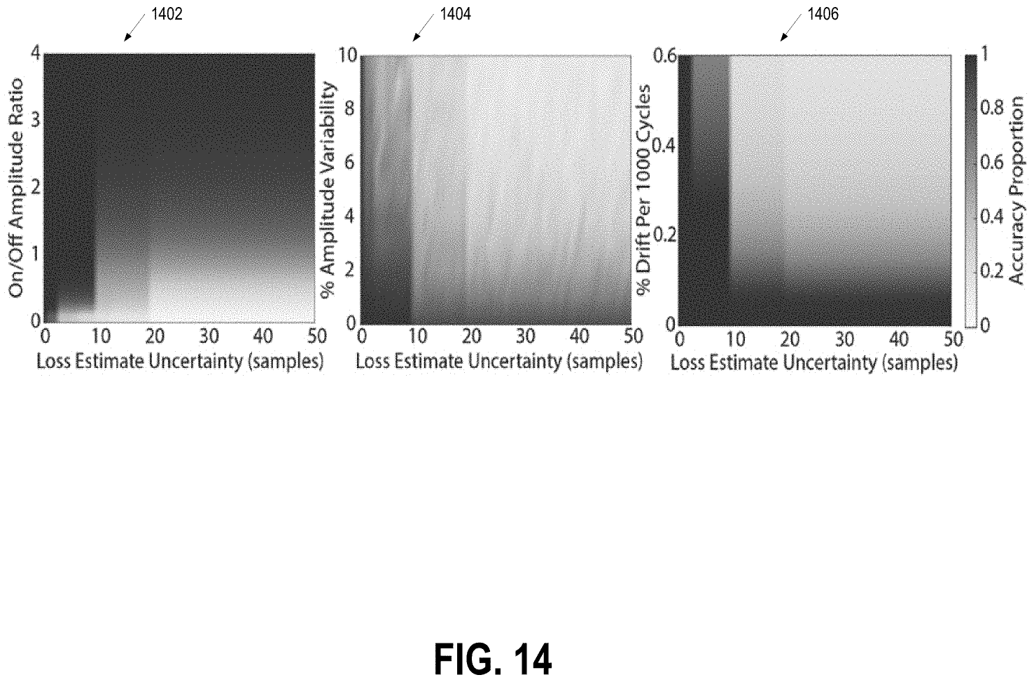

FIG. 14 shows the Monte Carlo simulated loss experiments measuring the accuracy of PELP estimates for stimulation amplitude ratio 1402, amplitude variability 1404, and drift 1406, as a function of loss estimate uncertainty. All three heat maps show discrete transitions in accuracy at uncertainty multiples of three samples. This occurs since for bilateral stimulation with a period of roughly 6.64 samples, estimated differences of multiples of three samples will be more closely overlapping. For constant uncertainty, accuracy increased smoothly for increasing amplitude ratio, decreasing amplitude variability, and decreasing drift. Keeping any of the three stimulation model parameters constant while varying uncertainty had little effect on accuracy with exception of the effects at multiples of three samples. Accuracy was near 100% for amplitude ratios above 0.2 for uncertainties less than 3 samples, amplitude ratios above 0.5 for uncertainties less than 9 samples, and amplitude ratios above 3 for uncertainties greater than 9 samples. Accuracy was near 100% for amplitude variabilities below 2% for uncertainties less than 9 samples. For uncertainties larger than 9, amplitude variability had to be near zero to maintain 100% estimate accuracy. Accuracy was near 100% for all drifts for uncertainties less than 3 samples, drifts less than 0.4% per 1000 cycles for uncertainties less than 9 samples, and drifts less than 0.1% per 1000 cycles for uncertainties greater than 9 samples.

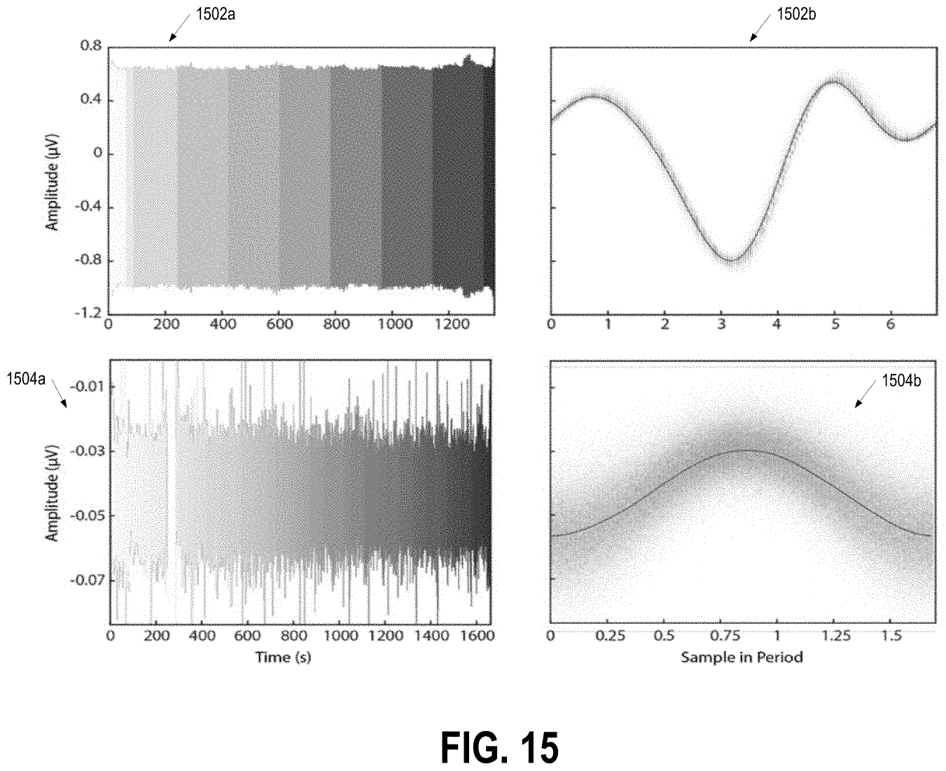

FIG. 15 shows LFP data from a behavioral task containing packet losses recorded using the SUMMIT RC+S in the clinic and at-home after estimation of losses using PELP. As shown in graphs 1502a and 1502b, data recorded in the clinic sampled at 1000 Hz contained 15 losses with a median size of 200 samples. Furthermore, as shown in graphs 1504a and 1504b, data recorded at home sampled at 250 Hz contained 121 losses with a median size of 17 samples. When overlapped on the timescale of the period of stimulation, all samples from both conditions were well consolidated about the stimulation waveform with no observable evidence of significant period drift indicating that losses were accurately accounted for.

Thus, streaming of intracranial electrophysiology data in the clinic and at home in ecologically valid environments may be essential for biomarker discovery in a variety of neurological disorders. Bidirectional implanted devices have enabled the acquisition of such datasets. However, data losses during wireless streaming hinder accurate analyses of neural signals. To address these issues, various aforementioned embodiments of the present disclosure describe novel and nonobvious systems and methods (e.g., PELP) to precisely estimate and account for data losses from implanted recordings where stimulation is on.

In some embodiments, PELP may be widely applicable to other stimulating devices capable of wireless data streaming. The stimulation model disclosed herein accurately accounts for the range of amplitude and variability parameters that could be expected for other implanted devices. For example, in recordings where sensing and stimulation occur on nearby contacts, stimulation amplitude can exceed the underlying neural signal by a factor of 10. For recordings where sensing and stimulation occur far apart or the stimulation harmonics fall within the transition band of an online low-pass filter, the amplitude ratio will be closer to 1.

Our recordings using the SUMMIT RC+S had amplitude ratios of 27 and 1.2 and estimate accuracy was consistent with predictions from the simulation. Pulse to pulse amplitude variability for the SUMMIT RC+S is well within the range of values where PELP was most accurate. Fluctuations in battery or the surrounding medium could influence amplitude on longer timescales. Instead of using the longest run for modeling stimulation in the entire recording, neighboring runs could be used for each prediction to account for slower drift in parameters or longer recordings. Similar considerations would also be effective if stimulation frequency drift or errors in period estimation occur.

Since PELP requires stimulation artifacts to be present in order to model the signal during data losses, the method may not necessarily be applicable for recordings where stimulation is off or significantly attenuated by online filters. In such circumstances, PELP may be complemented or replaced with less accurate methods utilizing packet timing metadata for loss estimation. In theory, stimulation could be applied below therapeutic amplitudes and can still be used for reconstruction using PELP although such modifications would be reasonable if the inevitable stimulation artifacts did not obscure neural signals of interest.

In some embodiments, PELP could be adapted to determine phase differences between discontinuous bursts of stimulation by minimizing mean squared error as a function of the stimulation phase offset between bursts. Such an approach could be useful for increasing the amount of data available to artifact removal methods relying on template subtraction.

By leveraging the periodicity of stimulation artifacts, PELP can produce highly accurate reconstructions of timing information from wirelessly transmitted neural data. PELP effectively accounts for the timing of missing data enabling the analysis of complex, naturalistic neural time series data from next-generation bidirectional implanted devices aiding in the development of novel therapeutic approaches.

### Period-Based Estimation of Electrical Stimulation Artifacts in the Presence of Phase Shifts

As previously discussed, various embodiments of the present disclosure describe systems and methods for period-based estimation of electrical stimulation artifacts. Such systems and methods may include the determination of a stimulation period of the artifact.

In some embodiments, estimation of electrical stimulation artifacts from waveform data may be made difficult or unfeasible due to a variety of factors. Such factors may include but are not limited to missing data of unknown duration (e.g., due to packet losses in wireless transmission combined with inaccuracies in device timing capabilities), device modulation between time segments of constant stimulation (e.g., caused by trials of interleaved stimulation-on and stimulation-off, and/or by the adjustment of device settings for clinical purposes), and device irregularities where the period of stimulation and/or sampling is momentarily different. Such factors may cause one or more phase shifts in the waveform data, further confounding the ability to estimate electrical stimulation artifacts from waveform data. Various embodiments of systems and methods for period-based estimation of electrical stimulation artifacts in the presence of phase shifts are thus described below. In some embodiments, such systems and methods may estimate these multiple phase shifts simultaneously with the estimation of the stimulation artifacts. Furthermore, such systems and methods may also or alternatively estimate the stimulation period of artifacts simultaneously with the estimation of the phase shifts from the waveform data.

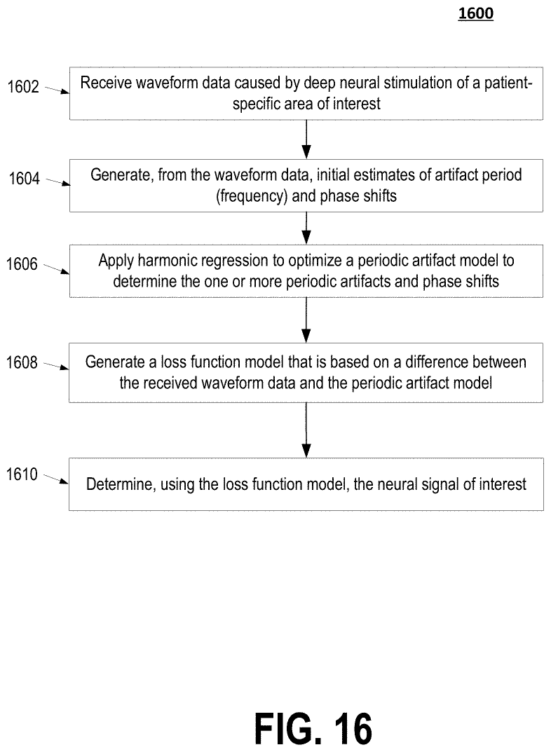

FIG. 16 is a flow diagram showing an example method 1600 of period-based estimation of electrical stimulation artifacts in the presence of phase shifts, according to a non-limiting embodiment of the present disclosure. One or more blocks of method 1600 may be performed by computer system 100 (e.g., via its processor 106 based on instructions stored in memory 104). In some embodiments, the computer system 100 may comprise a computing device, and may be referred to as computing device 100.

Method 1600 may begin with receiving waveform data caused by deep neural stimulation of a patient-specific area of interest (block 1602). For example, the patient-specific area of interest may comprise an area or section of a brain of a patient being treated, on which electrodes may be applied. The received waveform data may be characterized by a plurality of runs (e.g., segments). Each run (e.g., segment) may comprise a contiguous portion of the waveform data separated by a packet loss. Furthermore, the received waveform data may include (e.g., in addition to the underlying neural signal of interest), one or more periodic artifacts. Such artifacts may be estimated (e.g., based on their stimulation periods) using methods presented herein. Furthermore, as will be discussed in method 1600, the waveform data may be characterized as having multiple phase shifts. In some aspects, the waveform data may be received in real-time or near real-time. The received waveform data (e.g., observed signal) may be incorporated into a model (model of the received waveform data) in order to estimate and determine a periodic artifact, as will be discussed herein. An example of the model of the received waveform data, Sᵢ, may be as follows. In at least one embodiment, given n+1 segments of data, an i-th run (e.g., i-th segment) of the received waveform data (e.g., observed signal), Sᵢ, may be modeled as:

$${S}_{i} ( t ) = A ( t + \frac{{δ}_{i}^{*}}{{ξ}^{*}} ) + {B}_{i} ( t ) + {η}_{i} ( t ) , i = 0 , … , n ,$$

where δᵢ* represents the phase shift between the 0th and i-th run of the received waveform data, A represents a model of the periodic artifact based on a stimulation frequency ξ* (or stimulation period

$$( \frac{1}{{ξ}^{*}} ) )$$

of the periodic artifact and the phase shift (δᵢ*) of the plurality of phase shifts, Bᵢ(t) represents the neural signal of interest at the i-th run, and ηᵢ(t) represents noise at the i-th run. Since, the periodic artifact and the phase shifts may not be known when the waveform data is received, initial estimates for the periodic artifact and phase shifts may be generated (block 1604) as part of an initialization process described further below.

An objective of method 1600 may be to estimate and remove the model of the periodic artifact, A, from the model of the received waveform data at each run, Sᵢ. As a result, the underlying signal of interest that may be desired to be recovered may include: Bᵢ+ηᵢ, where i=0, . . . , n. In some embodiments, Sᵢ may be sampled at some collection of discrete times Tᵢ. Then, the following loss function can be used to reconstruct and remove the artifact: L(ξ,δᵢ,θ)=Σᵢ₌₀ⁿΣ_{tϵT}_{ᵢ}(Sᵢ(t)−a(t|ξ,δᵢ,θ))², where a(·|ξ,δᵢ,θ) represents the model of the periodic artifact, which may be based on: a stimulation frequency ξ (or stimulation period

$$( \frac{1}{ξ} ) )$$

of the periodic artifact, a phase shift δᵢ of the plurality of phase shifts of the received waveform data, and a set (possibly empty) of parameters θ for the periodic artifact, and where Sᵢ(t) is the model of the received waveform data at time t, and sampled at discrete times, Tᵢ. As shown in the loss function model, the model of the periodic artifact may be need to initially be determined. In some embodiments, the periodic artifact may be modeled by optimizing a parametric model of the periodic artifact using harmonic regression. In some aspects, a(·|ξ,δᵢ,θ) approximates, represents, and/or is approximated by the previously discussed

$$A ( \cdot + \frac{{δ}_{i}^{*}}{{ξ}^{*}} ) .$$

Thus, at block 1606, a periodic artifact model may be optimized (e.g., via harmonic regression) to estimate the periodic artifact and phase shifts from the received waveform data. For example, the following parametric equation may be used for periodic artifact model: a(t|ξ,δ,α₀,αₖ,βₖ,K)=α₀+Σₖ₌₁^{K}(αₖ cos(2πk(ξt+δ)+βₖ sin(2πk(ξt+δ))), where K may represent a specific number of harmonics, k. In some embodiments, the number of harmonics, K, applied during the harmonic regression must be chosen to be finite. Since the periodic artifacts model takes phase shifts into account, an optimization of the periodic artifacts model to determine or estimate the periodic artifact may also simultaneously determine or estimate the multiple phase shifts exhibited in the waveform data. Using the above described parametric model may allow for fast computations, thereby freeing up computing resources of computing device 100. Based on the model of the received waveform data, Si, and the aforementioned periodic artifact model to be optimized via harmonic regression, a loss model may be generated (block 1608). The loss function model (e.g., L(ξ,δᵢ,θ)=Σᵢ₌₀ⁿΣ_{tϵT}_{ᵢ}(Sᵢ(t)−a(t,ξ,δᵢ,θ))²) may be based on a difference between the received waveform data, Sᵢ(t), and the periodic artifact model a(t,ξ,δᵢ,θ))².

As previously discussed, in relation to FIG. 10, PELP may also use harmonic regression to estimate the periodic artifact. However, applying harmonic regression to the aforementioned periodic artifact model (e.g., the parametric equation discussed in block 1606) can accommodate multiple phase shifts. In contrast, the harmonic regression applied in PELP, as described in relation to FIG. 10, estimates only a single unknown phase from two segments (n=1), and may not necessarily consider simultaneous inference of the model parameters. Instead, PELP, as described in FIGS. 10-15 may initially estimate the artifact and its frequency from a single segment (e.g., using methods of determining stimulation period described in FIG. 4) and may then select, among a small finite collection of candidate phases corresponding to an unknown number of missing samples. Furthermore, the previously described PELP process may be repeated sequentially to handle multiple phases (n>1). In contrast, the process shown in FIG. 16 is not necessarily sequential; since it is based on an optimization of the loss function model.

In some embodiments, the aforementioned parametric model for the periodic artifact, a(t|ξ,δ,α₀,αₖ,βₖ,K) (“periodic artifact model”) may be used to remove the periodic artifact from the received waveform data (modeled as Si) using harmonic regression. For example, harmonic regression may be applied to optimize the periodic artifact model a(t|ξ,δ,α₀,αₖ,βₖ,K) to estimate one or more periodic artifacts from the received waveform data (block 1606). For fixed frequencies of artifacts and phase shifts (ξ,δ), the harmonic regression may be equivalent to minimizing the loss function model, L(ξ,δᵢ,θ) with respect to θ={α₀,αₖ,βₖ,k=1, . . . , K}. Thereafter, the previously discussed loss function model, L(ξ,δᵢ,θ)=Σᵢ₌₀ⁿΣ_{tϵT}_{ᵢ}(Sᵢ(t)−a(t|ξ,δᵢ,θ))², may be generated based on the model of the received waveform data Sᵢ(t) and the optimized periodic artifact model a(t|ξ,δ,α₀,αₖ,βₖ,K) (block 1608). The loss function model may be optimized (e.g., via application of harmonic regression to the periodic artifact model) determine the neural signal of interest (block 1610). Moreover, since the model of the received waveform data Sᵢ(t) and the periodic artifact model a(t|ξ,δ,α₀,αₖ,βₖ,K) accommodate for a plurality of unknown phase shifts δᵢ, optimizing the loss function model may also simultaneously estimate or determine the plurality of phase shifts, as previously discussed in block 1610).

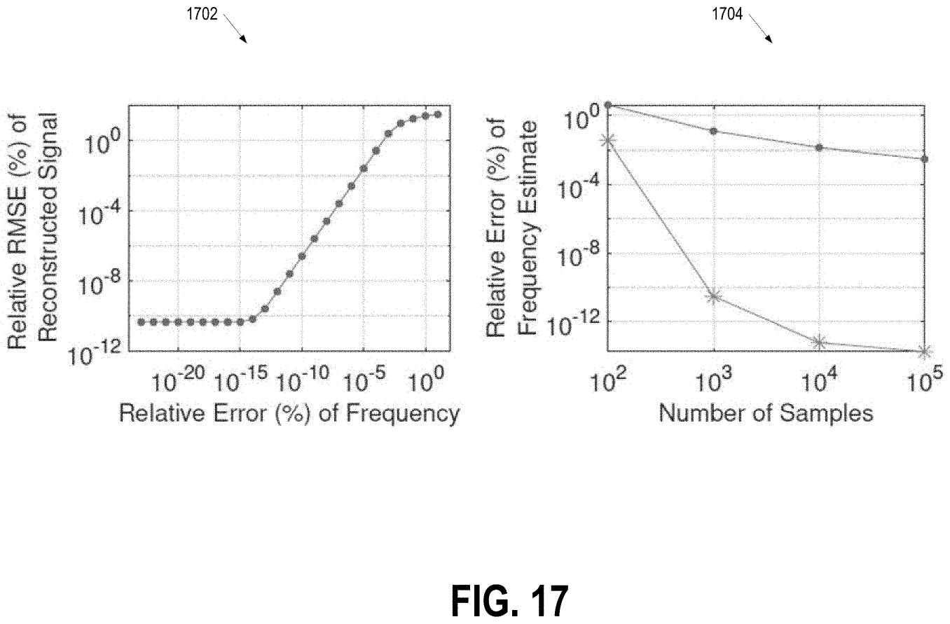

FIG. 17 shows graphs illustrating the effectiveness of the method of period-based estimation of electrical stimulation artifacts in the presence of phase shifts, according to a non-limiting embodiment of the present disclosure. Graph 1702 shows a relative error of frequency plotted over a relative root mean square error (RMSE) of reconstructed signal using harmonic regression. Each point represents the average over 40 trials. For each trial, harmonic regression was used to fit the observed signal with a sinusoid with 5 harmonics and a given frequency estimate. As shown in graph 1702, harmonic regression may require accurate estimates of the frequency ξ. For example, using harmonic regression, 0:001% relative error in the frequency estimate, on average, corresponds to a relative root mean squared error (RMSE) in the reconstructed signal Sᵢ−a(·|ξ,δᵢ,θ) of almost 10%. A relative RMSE may be defined as:

relative RMSE={square root over (Σᵢ₌₁^{N}(f(tᵢ)−\hat{f}(tᵢ))²/Σᵢ₌₁^{N}f(tᵢ)²)}, where f is the true signal of interest, \hat{f} is the estimated signal, and tᵢ are the sample times. It is to be appreciated that even somewhat small errors in the frequency may lead to large reconstruction errors.

Many periodic artifact removal methods rely on the discrete Fourier transform (DFT) to estimate the frequency. However, DFT-based methods generally cannot handle phase shifts and, even segment-wise, are limited in their accuracy for frequency estimation. For example, consider the following simple DFT-based frequency detection method, where the artifact frequency is estimated as the frequency that maximizes the energy (magnitude squared of the DFT) of the observed signal. Graph 1704 of FIG. 17 shows the number of samples versus a relative error of the frequency estimated using a DFT-based method (shown in blue at the top) or using the method discussed in FIG. 16 (shown in red at the bottom). Each point represents the average over 10 trials. In both plots, each trial uses an observed signal sampled at 1000 Hz that consists only of an artifact modeled as a(t|ξ,δ,α₀,αₖ,βₖ,K) with no phase shifts and K=5. As shown in graph 1704 of FIG. 17, even in the case where the observed signal only consists of the periodic artifact and the sampling rate is relatively high (fs=1000 Hz), this DFT-based method is unable to accurately estimate the artifact frequency when the frequency does not lie on the DFT grid

$$\{ i \frac{N}{{f}_{s}} : i = 0 , … , \frac{N}{2} - 1 \} ,$$

which may be the case for most real-life applications. Specifically, even with 10⁵ samples, this method may achieve, on average, greater than 0:001% relative error in the frequency estimate, which, as discussed above, is insufficiently accurate for harmonic regression.

In contrast, graph 1704 also shows that the method discussed herein (e.g., in FIG. 16) for period-based estimation of stimulation artifacts in the presence of phase shifts achieves much more accurate frequency estimates, e.g., close to machine precision with 10⁵ samples.

In some embodiments, the iterative periodic artifact removal methods and systems described herein can accurately estimate the artifact frequency in the presence of unknown phase shifts and may involve few tuning parameters and relatively simple computations. An example system and method is further described below. First, the artifact removal process is further described, which removes the artifact by estimating the frequency and phase shifts while jointly fitting the artifact using harmonic regression. Second, an initialization process is further described, which finds an initial estimate of the frequency and phase shifts via an energy maximization method.

For n+1 runs (e.g., segments of waveform data Sᵢ), where i=0; . . . , n, a model for the waveform data, Sᵢ, may be defined as follows:

$${S}_{i} ( t ) = A ( t + \frac{{δ}_{i}^{*}}{{ξ}^{*}} ) + {B}_{i} ( t ) + {η}_{i} ( t ) , i = 0 , … , n$$

(e.g., as previously discussed), and δᵢ* may represent the (unknown) true phase shift between the 0-th and i-th runs (e.g., segments) (by convention, δ₀*=0). The artifact may be assumed to be represented by a(t|ξ,δ,α₀,αₖ,βₖ,K) (e.g., as previously discussed), where the true fundamental frequency ξ*, the true amplitudes α₀*,αₖ*,βₖ*, and the true number of harmonics K are unknown. It may be assumed for this process that the energy of the artifact at its fundamental frequency and harmonics can be sufficiently larger than the energy of the brain at those frequencies (or at the frequencies that they are aliased to). In some embodiments, there may not be access to the continuous signals and there may only be access to each Si at some discrete sample times. While these sample times may be irregularly spaced, the process considers only uniformly spaced sample times as most DBS devices typically use uniformly spaced sampling based on relatively stable system clocks. Thus, it is assumed that the i-th segment is sampled at the times

$$\{ \frac{j}{{f}_{s}} : j = 0 , … , {N}_{i} - 1 \} ,$$

where fₛ is the sampling rate.

Based on the assumed form of the artifact a(t|ξ,δ,α₀,αₖ,βₖ,K), harmonic regression may be is a natural choice for removing the artifact given an estimate of the frequency. However, as shown in FIG. 17 (e.g., graph 1702), small perturbations in the frequency estimate can result in large errors in the signal reconstructed by harmonic regression. To mitigate this issue, the artifact removal process may involve fitting the observed signal using harmonic regression while jointly estimating the frequency and phase shifts. Specifically, the artifact may be removed by solving the following optimization problem:

$${min}_{ω , {δ}_{i} , i = 1 , … , n} ℊ ( ω , {δ}_{i} , … , {δ}_{n} ) ,$$

where the objective function g is defined by:

$$ℊ ( ω , {δ}_{i} , … , {δ}_{n} ) = {min}_{{α}_{0} , {α}_{k} , {β}_{k}} {\sum }_{i = 0}^{n} {\sum }_{j = 0}^{{N}_{i} - 1} {( {R}_{i} ( \frac{j}{{f}_{s}} , {α}_{0} , {α}_{k} , {β}_{k} ) )}^{2} ,$$

where the i-th segment of the recovered signal can be modeled as: Rᵢ(t, α₀,αₖ,βₖ)=Sᵢ(t)−a(t|ω, δᵢ, α₀, αₖ, βₖ, \hat{K}), where a(t|ω, δᵢ, α₀, αₖ, βₖ, \hat{K}) is defined by a(t|ξ,δ,α₀,αₖ,βₖ,K). For each fixed (ω, δᵢ, . . . , δₙ), g(ω, δᵢ, . . . , δₙ) is the result of using harmonic regression to remove a periodic artifact of the form a(t|ξ,δ,α₀,αₖ,βₖ,K) with K=\hat{K} harmonics, fundamental frequency ξ*=ω, and phase shifts δᵢ from the 0-th observed segment S₀. In some embodiments, the number of harmonics to fit \hat{K} may effectively be the only parameter that needs to be tuned. Minimizing α₀ in

$$ℊ ( ω , {δ}_{i} , … , {δ}_{n} ) = {min}_{{α}_{0} , {α}_{k} , {β}_{k}} {\sum }_{i = 0}^{n} {\sum }_{j = 0}^{{N}_{i} - 1} {( {R}_{i} ( \frac{j}{{f}_{s}} , {α}_{0} , {α}_{k} , {β}_{k} ) )}^{2}$$

may mean that the recovered signal will have a mean of 0. Including this amplitude may be optional, and if α₀ is omitted in Rᵢ(t,α₀,αₖ,βₖ), then the recovered signal may have a nonzero mean, but the reconstructed artifact may necessarily have a mean of 0. Furthermore, each fixed (ω, δᵢ, . . . , δₙ), g(ωm δᵢ, . . . , δₙ) can then be computed up to numerical precision errors. Specifically, the gradient of the objective function in

$$ℊ ( ω , {δ}_{i} , … , {δ}_{n} ) = {min}_{{α}_{0} , {α}_{k} , {β}_{k}} {\sum }_{i = 0}^{n} {\sum }_{j = 0}^{{N}_{i} - 1} {( {R}_{i} ( \frac{j}{{f}_{s}} , {α}_{0} , {α}_{k} , {β}_{k} ) )}^{2}$$

may be set to zero to solve the resulting linear system using an appropriate numerical linear algebra solver.

For example, Newton's descent method may be used to solve for the optimization problem

$${min}_{ω , {δ}_{i} , i = 1 , … , n} ℊ ( ω , {δ}_{i} , … , {δ}_{n} ) .$$

in some aspects, the numerical complexity of the above described artifact removal process may be dominated by the computation of the descent direction, which may requires solving for an (n+1)×(n+1) and a \hat{K}×\hat{K} linear system. Thus, the numerical complexity of the algorithm is O(n³+\hat{K}³). In practice, both n and \hat{K} may be relatively small since the data can always be segmented so as not to consider too many gaps at a time and many DBS devices have built-in low-pass filters that effectively band limit the recorded signals. Newton's descent can also be applied to this problem since g can be twice differentiable. Also while g may not be a convex function, g may be locally convex at the global minimizer. Hence, the initialization for the artifact removal process may be crucial for convergence. An initialization process for the artifact removal process is thus described. Based on numerical experiments, the initialization process may typically have relatively short computational runtime and may provide sufficiently accurate initializations for the artifact removal process.

The initialization process locally maximizes the energy of the observed signal using discrete samples but without being constrained to frequencies on the DFT grid. A Fourier transform for the initialization process may be defined as follows:

$$F ( ω , {δ}_{1} , … , {δ}_{n} ) = {\int }_{0}^{\frac{{N}_{0}}{{f}_{s}}} {S}_{0} ( t ) {e}^{- 2 π i ω t} dt + {\sum }_{l = 1}^{n} {\int }_{0}^{\frac{{N}_{0}}{{f}_{s}}} {S}_{l} ( t ) {e}^{- 2 π i ( ω t + {δ}_{l} )} dt .$$

The above Fourier transform aligns all of the segments Sₗ (l=0, 1, . . . , n) in time to be in phase with a single wave e^{-2πiωt}. Furthermore, the energy may be defined as:

E(ω,δ₁, . . . ,δₙ=| [symbol] (ω,δ₁, . . . ,δₙ)|²,

By straightforward calculation, one can easily verify that E is twice differentiable and nonconcave. The True frequency ξ* and true phase shifts δᵢ* may be recovered by solving the following optimization problem:

$${max}_{ω , {δ}_{1} , … , {δ}_{n}} E ( ω , {δ}_{1} , … , {δ}_{n} ) .$$

Note that for fixed ω,δⱼ,j≠i, the function δᵢ→ [symbol] (ω,δ₁, . . . ,δₙ) (and hence, the function δᵢ→E(ω, δ₁, . . . , δₙ)) is 1-periodic. Thus, solving

$${max}_{ω , {δ}_{1} , … , {δ}_{n}} E ( ω , {δ}_{1} , … , {δ}_{n} )$$

may only recover the phase shifts i up to a multiple of 1. Moreover, since E is nonconcave, the maximizers of (11) may not necessarily be unique.

In the initialization process,

$${max}_{ω , {δ}_{1} , … , {δ}_{n}} E ( ω , {δ}_{1} , … , {δ}_{n} )$$

can be solved numerically by applying a modified Newton's ascent method with backtracking line search and random initialization.

Since there may only be access to discrete samples (and not the continuous signals), the integrals in the definitions of the energy E, its gradient ∇E, and its Hessian ∇E² may be approximated using trapezoidal rule. The computational effort for each iteration of the initialization process may be proportional to (n+1)³. Typically, the data can always be segmented so that n is relatively small. Additionally, the implementation of the initialization process can be made more efficient by parallelizing the computations for each random initialization.

Thus, a periodic artifact removal process (and a corresponding initialization process) has been described herein that is able to accurately recover underlying signals in the presence of unknown phase shifts (e.g., missing data of unknown length), and in some cases where the stimulation frequency exceeds the Nyquist frequency. The disclosed processes effectively require only one tuning parameter—the number of harmonics \hat{K} to fit. Choosing k too small may cause an underfitting of the artifact, while choosing a \hat{K} that is too large may cause an overfitting of the artifact. Thus, the parameter \hat{K} can be easily interpreted and could be roughly estimated by counting the number of bands corresponding to the harmonics of the artifact that are visible in a spectrogram of the observed signal.

Herein, a computer-readable non-transitory storage medium or media may include one or more semiconductor-based or other types of integrated circuits (ICs) (e.g., field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, features, functions, operations, blocks, or steps, any of these embodiments may include any combination or permutation of any of the components, elements, features, functions, operations, blocks, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

All of the disclosed methods and procedures described in this disclosure can be implemented using one or more computer programs or components. These components may be provided as a series of computer instructions on any conventional computer readable medium or machine readable medium, including volatile and non-volatile memory, such as RAM, ROM, flash memory, magnetic or optical disks, optical memory, or other storage media. The instructions may be provided as software or firmware, and may be implemented in whole or in part in hardware components such as ASICs, FPGAs, DSPs, or any other similar devices. The instructions may be configured to be executed by one or more processors, which when executing the series of computer instructions, performs or facilitates the performance of all or part of the disclosed methods and procedures.

It should be understood that various changes and modifications to the examples described here will be apparent to those skilled in the art. Such changes and modifications can be made without departing from the spirit and scope of the present subject matter and without diminishing its intended advantages. It is therefore intended that such changes and modifications be covered by the appended claims.

1. A system for reconstruction and removal of electrical stimulation artifact, the system comprising:
an intracranial electroencephalography (iEEG) device;
one or more processors; and
memory storing instructions that, when executed by the one or more processors, cause the one or more processors to:
receive, from the iEEG device, waveform data caused by a deep brain stimulation of a patient-specific area of interest;
determine, based on a regularization of the waveform data, a stimulation period relative to a sampling rate;
identify, based on the waveform data and the stimulation period, a stimulation artifact;
subtract the identified stimulation artifact from the received waveform data; and
generate a filtered waveform data indicating an absence of the stimulation artifact.

2. The system of claim 1, wherein the instructions, when executed, cause the one or more processors to determine the stimulation period relative to the sampling rate by:
performing one or more iterations of:
selecting, from the waveform data, a candidate stimulation period;
estimating, based on the candidate stimulation period, a waveform template; and
quantifying a deviation of the candidate stimulation period from the waveform template; and
identifying, as the stimulation period relative to the sampling rate, the candidate period associated with a quantified deviation that satisfies a threshold.

3. The system of claim 2, wherein the instructions, when executed, cause the one or more processors to determine the stimulation period relative to the sampling rate by:
identifying, between two contiguous time segments of the waveform data, one or more phase shifts in the waveform data;
estimating, based on the one or more phase shifts, a number of time points in one or more missing packets of unknown duration; and
realigning, based on the number of time points in the one or more missing packets, the waveform data,
wherein the stimulation period is based on the realigned waveform data.

4. The system of claim 1, wherein the instructions, when executed, cause the one or more processors to identify the stimulation artifact by applying Nadaraya-Watson kernel regression and a linear filter to the waveform data and the stimulation period.

5. The system of claim 1, wherein the instructions, when executed, cause the one or more processors to identify the stimulation artifact by:
recording a plurality of proximal waveform data received proximal to one or both of a time or a stimulation phase of the received waveform data;
averaging the recorded proximal waveform data; and
identifying, based on the averaged recorded proximal waveform data, the stimulation artifact.

6. The system of claim 5, wherein the instructions, when executed, further cause the system to:
apply one or more design parameters to average the recorded proximal waveform data and to identify the stimulation artifact, wherein the one or more design parameters comprises:
a first design parameter that controls a quantity of proximal waveform data to be averaged;
a second design parameter that controls a quantity of proximal waveform data to skip; or
a third design parameter that controls a minimum duration for a candidate simulation period.

7. The system of claim 1, wherein the instructions, when executed, causes the one or more processors to receive the waveform data by:
recording, in real-time and via electrodes connected to the patient-specific area of interest, an intracranial electroencephalography (iEEG) scan; and
wherein the instructions, when executed, causes the one or more processors to generate the filtered waveform data in real-time.

8. A method of period estimation of electrical stimulation artifacts in the presence of phase shifts, the method comprising:
receiving, by a computing device having a processor, waveform data caused by brain stimulation of a patient-specific area of interest,
wherein the waveform data comprises:
a plurality of runs,
a plurality of phase shifts, and
a periodic artifact;
wherein each run indicates a contiguous portion of the waveform data separated by a packet loss;
generating a model of the received waveform data based on:
a neural signal of interest and a model of the periodic artifact, wherein the model of the periodic artifact is based on a stimulation period of the periodic artifact and a phase shift of the plurality of phase shifts;
applying harmonic regression to optimize the model of the periodic artifact;
determining simultaneously, using an optimization of a loss model, the plurality of phase shifts and the neural signal of interest, wherein the loss model is based on the model of the received waveform data and an optimized model of the periodic artifact.

9. The method of claim 8, wherein the model of the received waveform data comprises:
$${S}_{i} ( t ) = A ( t + \frac{{δ}_{i}^{*}}{{ξ}^{*}} ) + {B}_{i} ( t ) + {η}_{i} ( t ) , i = 0 , … , n ,$$
wherein δᵢ* comprises the phase shift, of the plurality of phase shifts, between the 0th and i-th run of the plurality of runs,
wherein A is the model of the periodic artifact based on a stimulation period
$$( \frac{1}{{ξ}^{*}} )$$
of the periodic artifact and the phase shift (δᵢ*) of the plurality of phase shifts,
wherein Bᵢ(t) is the neural signal of interest at the i-th run, and
wherein ηᵢ(t) is noise at the i-th run.

10. The method of claim 8, wherein the loss model comprises:
$$L ( ξ , {δ}_{i} , θ ) = {\sum }_{i = 0}^{n} {\sum }_{t ∈ {T}_{i}} {( {S}_{i} ( t ) - a ( t \vert ξ , {δ}_{i} , θ ) )}^{2} ,$$
wherein α(·|ξ,δᵢ,θ) is the optimized model of the periodic artifact,
wherein the optimized model of the periodic artifact is based on:
a stimulation frequency ξ of the periodic artifact,
the phase shift δᵢ of the plurality of phase shifts, and
one or more parameters for the periodic artifact; and
wherein Sᵢ(t) is the model of the received waveform data at time t, and sampled at discrete times, Tᵢ.

11. The method of claim 10, wherein the model of the periodic artifact to which harmonic regression is applied comprises:
a(t|ξ,δ,α₀,αₖ,βₖ,K)=α₀+Σₖ₌₁^{K}(αₖ cos(2πk(ξt+δ))+βₖ sin(2πk(ξt+δ))), wherein K is a finite number of harmonics, k.

12. The method of claim 8, further comprising:
applying, to the model of the received waveform data, an initialization process to locally maximize an energy of the waveform data; and
removing the periodic artifact from the waveform data to generate a filtered waveform data indicating an absence of the stimulation artifact.

13. The method of claim 8, further comprising, prior to applying the harmonic regression:
generating an initial estimate for the stimulation period of the periodic artifact and an initial estimate for the plurality of phase shifts.

14. A method for periodic estimation of lost packets, the method comprising:
receiving, by a computing device having a processor, waveform data caused by brain stimulation of a patient-specific area of interest,
wherein the waveform data comprises a plurality of runs,
wherein each run indicates a contiguous portion of the waveform data separated by a packet loss;
estimating, based on the plurality of runs, one or more phase shifts in the waveform data;
determining, by the computing device and based on a regularization of the waveform data, a stimulation period relative to a sampling rate;
applying, by the computing device, based on the stimulation period, a harmonic regression model to a longest run of the plurality of runs; and
determining, based on the harmonic regression model, a maximal size of packet loss for the waveform data.

15. The method of claim 14, wherein applying the harmonic regression model to the longest run of the plurality of runs comprises:
determining, by the computing device, a root mean squared error (RMSE) between the longest run and a plurality of candidate waveforms; and
identifying, based on the RMSE, a candidate waveform, of the plurality of candidate waveforms, that satisfies a RMSE threshold.

16. The method of claim 14, wherein determining the stimulation period relative to the sampling rate comprises:
identifying, between two runs of the waveform data, one or more phase shifts in the waveform data;
estimating, based on the one or more phase shifts, a number of time points in one or more missing packets of unknown duration; and
realigning, based on the number of time points in the one or more missing packets, the waveform data,
wherein the stimulation period is based on the realigned waveform data.

17. The method of claim 14, further comprising:
applying, by the computing device, a second harmonic regression model to a second run of the plurality of runs.

18. The method of claim 17, wherein the optimal size of the packet loss for the waveform data is further based on the second harmonic regression model.

19. The method of claim 14, further comprising:
adding, by the computing device, a drift factor to the simulation period.

20. The method of claim 14, wherein receiving the waveform data caused by the deep brain stimulation of the patient-specific area of interest comprises:
recording, via electrodes connected to the patient-specific area of interest, an intracranial electroencephalography (iEEG) scan.
